(19) United States
(12) Patent Application Publication
(10) Pub. No.: US 2022/0374739 A1
(43) Pub. Date: Nov. 24, 2022

(54) Predicting Response to Stimulus

(71) Applicant: Optios, Inc., San Diego, CA (US)

(72) Inventors: Lucas Parra, Brooklyn, NY (US); Jacek Dmochowski, Montclair, NJ (US)

(21) Appl. No.: 17/745,820

(22) Filed: May 16, 2022

Related U.S. Application Data
(63) Continuation of application No. 14/433,279, filed on Apr. 2, 2015, now abandoned.
(60) Provisional application No. 61/822,382, filed on May 12, 2013.
(60) Provisional application No. 61/712,430, filed on Oct. 11, 2012.

(51) Int. Cl. G06N 5/04; G06N 20/00; A61B 5/00; G16H 50/70; A61B 5/377

(52) U.S. Cl. CPC G06N 5/04; G06N 20/00; A61B 5/7275; G16H 50/70; A61B 5/377; G06Q 30/0242

(57) ABSTRACT

A method of predicting response to a sensory stimulus includes, with a processor, automatically receiving behavioral data representing the response of a first population of subjects to a reference stimulus. Data representing the neurological responses of a second, different population of subjects to the reference sensory stimulus are received and processed to provide group-representative data indicating commonality between the neurological responses of at least two members of the second population. A mapping from the group-representative data to the received behavioral data is produced. Test data representing the neurological responses of a third population of subjects to a test sensory stimulus are received and processed to provide test group-representative data indicating commonality between the neurological responses to the test sensory stimulus of at least two members of the third population. The mapping is applied to the test group-representative data to provide predicted behavioral data.

### CROSS-REFERENCE TO RELATED APPLICATIONS

This application is a continuation of U.S. application Ser. No. 14/433,279 nationalized Apr. 2, 2015, which is a National Phase application of International App. No. PCT/US 2013/064474 filed Oct. 11, 2013, which claims the benefit of U.S. Prov. App. No. 61/822,382 filed May 12, 2013 and U.S. Prov. App. No. 61/712,430 filed Oct. 11, 2012. The entirety of each of the above applications is hereby incorporated by reference.

### FIELD

The present application relates to analysis of neurological data, and particularly to correlating neurological responses with stimuli.

### BACKGROUND

“Neuromarketing” is the employment of neuroimaging tools (mainly functional magnetic resonance imagery (fMRI) or electroencephalography (EEG)) to measure the neural response of a consumer presented with a stimulus in order to infer or predict the overall consumer base reaction to a particular product or service offering. Many stimuli involved in neuromarketing efforts possess a narrative structure: an ordered, connected sequence of events. Examples of these are: advertisements, television series episodes, motion pictures, educational videos and lectures, audiobooks, musical arrangements, and political speeches. These stimuli possess a temporal trajectory, and human brains are adapted to perceive, parse, track, and form ideas about such stimuli.

Past neuromarketing efforts have sought to identify brain regions (typically voxels in the magnetic resonance imagery space) which correlate with a certain cognitive or behavioral response. For example, elevated activity in the orbitofrontal cortex (OFC) has been implicated in pleasure and reward processing. As such, a typical neuromarketing study will measure activity in the OFC, and attempt to use these measurements to predict the ability of the proposed product or service to elicit pleasure during consumption by the general population. However, it is becoming increasingly more apparent that complex tasks such as enjoying a musical arrangement or following a movie scene involve an interplay among several distinct brain areas. Thus, it is suboptimal to utilize the neural response at a single, pre-defined brain location as a marker of consumption. An approach of correlating brain activity with various behaviors is not feasible because of the dimensionality of the problem and the level of noise in the neural signals, i.e. learning an arbitrary mapping from high-dimensional neural data to behavior is bound to fail due to high noise and limited data available. It is interesting to note that, when presented with a stimulus, the recorded neural activity reflects not only the response of the user to that stimulus, but also ongoing activity which is not specific to the stimulus and is uninformative from a neuromarketing standpoint. This stimulus-decoupled activity may in fact be as powerful (signal amplitude) as the desired sensory-driven response.

Recent work demonstrated that natural stimuli elicit reliable responses within and across individuals using fMRI and the electrocorticogram (EcoG) signals. High levels of inter-subject correlation have been linked to successful memory encoding and successful communication between individuals; they are increased for scenes marked by high arousal and negative valence, and are strongest for familiar and naturalistic events.

Hassan proposed to use intra- or inter-subject correlations in neural activity to estimate how engaging a stimulus is (U.S. patent application Ser. No. 12/921,076).

Reference is made to the following:
U.S. Pat. No. 6,099,319 to Zaltman et al. (Aug. 8, 2000);
U.S. Pat. No. 6,315,569 to Zaltman et al. (Nov. 13, 2001);
U.S. Pat. No. 8,209,224 to Anantha Pradeep, Robert T. Knight, Ramachandran Gurumoorthy entitled “Intracluster content management using neuro-response priming data;”
U.S. Publication No. 2011/0085700 by Hans C. Lee entitled “Systems and Methods for Generating Bio-Sensory Metrics,” U.S. patent application Ser. No. 12/835,714;
U.S. Publication No. 2011/0161011 by U. Hasson, R. Malach, and D. Heeger entitled “Computer-accessible medium, system and method for assessing effect of a stimulus using intersubject correlation,” U.S. patent application Ser. No. 12/971,076;
U.S. Publication No. 2011/0301431 by Greirius et al. entitled “Methods of classifying cognitive states and traits and applications thereof,” U.S. patent application Ser. No. 13/153,465;
U.S. Pat. No. 8,082,215 to E. K. Y. Jung et al. entitled “Acquisition and particular association of inference data indicative of inferred mental states of authoring users;” each of which is incorporated herein by reference.

Reference is also made to the following:
D. Ariely and G. S. Berns, “Neuromarketing: the hope and hype of neuroimaging in business.” Nature Neuroscience Reviews, 11 (2011).
F. Babiloni, “Consumer neuroscience: a new area of study for biomedical engineers.” IEEE Pulse Magazine, May/June 2012, pp. 21-23.
T. A. Hare, C. Camerer, and A. Rangel, “Self-control in decision-making involves modulation of the vmPFC valuation system.” Science 324 (2009): 646-648
B. Knutson, S. Rick, G. E. Wimmer, D. Prelec, and G. Loewenstein, “Neural predictors of purchases.” Neuron, 53 (2007): 147-156
H. Plassmann, J. O'Doherty, and A. Rangel, “Orbitofrontal cortex encodes willingness to pay in everyday economic transactions.” J. Neurosci. 27 (2007): 9984-9988
G. Vecchiato et al., “On the Use of EEG or MEG Brain Imaging Tools in Neuromarketing Research.” Computational Intelligence and Neuroscience Volume 2011 (2011), Article ID 643489, 12 pages, doi:10.1155/2011/643489
G. Vecchiato, W. Kong, A. G. Maglione, and D. Wei, “Understanding the impact of TV commercials.” IEEE Pulse Magazine, May/June 2012, pp. 42-47

### SUMMARY

However, prior schemes do not consider using intra and inter-subject correlation to predict various and diverse behavioral responses of a large audience. Herein are listed a wide variety of behaviors that may be of interest; prior-art measures are not effective at predicting these behaviors. In particular, prior schemes using a single measure of correlation cannot provide predictions in such diverse areas. The prior art does also not describe combining neural signals with additional information such as properties of the stimulus or behavioral responses from a group of individuals to predict behavioral responses.

According to an aspect of the invention, there is provided a method of predicting response to a sensory stimulus, the method comprising automatically performing the following steps using a processor:

receiving behavioral data representing the response of a first population of subjects to a reference sensory stimulus;
receiving neurological data representing the neurological responses of a second, different population of subjects to the reference sensory stimulus;
processing the received neurological data to provide group-representative data indicating commonality between the neurological responses of at least two members of the second population of subjects;
producing a mapping from the group-representative data to the received behavioral data;
receiving test neurological data representing the neurological responses of a third population of subjects to a test sensory stimulus;
processing the test neurological data to provide test group-representative data indicating commonality between the neurological responses to the test sensory stimulus of at least two members of the third population of subjects; and applying the mapping to the test group-representative data to provide data representing a predicted behavioral response to the test sensory stimulus.

This brief description is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

The above and other objects, features, and advantages of the present invention will become more apparent when taken in conjunction with the following description and drawings. Identical reference numerals have been used, where possible, to designate identical features that are common to the figures.

FIG. 1 shows a schematic representation of a prediction approach for predicting audience response from aggregated neural responses;

FIG. 2 shows a flowchart illustrating an exemplary method for collecting neural responses on a group of individuals to predict viewership or other audience behavioral responses;

FIG. 3 shows an example of prediction accuracy as a function of temporal aperture;

FIG. 4 shows viewership data and predictions of minute-by-minute viewership ratings from the amount of neural response reliability observed in a small sample of test subjects for the example of FIG. 3;

FIG. 5 shows an example of predicting the frequency of tweets;

FIGS. 6-9 show an example of the prediction of audience behavioral response to different video content;

FIG. 10 depicts projections of the correlated neural activity on the scalp for the top three correlation-maximizing components of three different stimuli;

FIG. 11 shows within-subject correlation over time for a motion-picture stimulus;

FIG. 12 is a graph of the percentage of time windows of various motion-picture stimuli that exhibit significant correlation;

FIG. 13 is graph organized as FIG. 12 and comparing percent-significant-correlation for a motion-picture stimulus with that measure for the same motion picture with its scenes rearranged;

FIG. 14 depicts the scalp projections of the maximally-correlated components for a motion-picture stimulus on two successive viewings;

FIG. 15 depicts time-resolved correlation coefficients averaged across subject-pairs for each of two successive viewings;

FIG. 16 is a graph organized as FIG. 12 and comparing percent-significant-correlation for two successive viewings of a motion-picture stimulus;

FIG. 17 shows results of a comparison of instantaneous power at several nominal EEG frequency bands (collapsed across subjects and viewings) during times of high within-subject correlation with that observed during low-correlation periods;

FIGS. 18-20 show sources of correlated neural activity for respective components; and

FIG. 21 is a high-level diagram showing components of a data-processing system.

The attached drawings are for purposes of illustration and are not necessarily to scale.

### DETAILED DESCRIPTION

Throughout this description, some aspects are described in terms that would ordinarily be implemented as software programs. Those skilled in the art will readily recognize that the equivalent of such software can also be constructed in hardware, firmware, or micro-code. Because data-manipulation algorithms and systems are well known, the present description is directed in particular to algorithms and systems forming part of, or cooperating more directly with, systems and methods described herein. Other aspects of such algorithms and systems, and hardware or software for producing and otherwise processing signals or data involved therewith, not specifically shown or described herein, are selected from such systems, algorithms, components, and elements known in the art. Given the systems and methods as described herein, software not specifically shown, suggested, or described herein that is useful for implementation of any aspect is conventional and within the ordinary skill in such arts.

It has been determined that inter-subject and intra-subject correlations in the EEG capture engagement of an audience with a stimulus. These stimuli possess a temporal trajectory, and our brains have been evolutionarily tuned to perceive, parse, track, and form ideas about such stimuli. The technology proposed here leverages this exquisite processing capability in a system which tracks and indexes ongoing state changes in real-time.

Various aspects described herein spatially filter across multiple sensors to compute measurements that reflect the contributions of multiple brain regions forming distributed but coherent networks, i.e., there is no limitation imposed by a-priori information on the association of specific brain areas or neural signals with specific behaviors. In various aspects, the reliability of these distributed patterns of neural activity across multiple subjects and within subjects are used as a key feature that carries predictive information as to the general audience's behavioral responses, e.g., to the viewership tendencies of the population from which they are sampled. Various aspects extract signals that are reliably reproduced within subjects and agree across subjects and use those signals as a mechanism of dimensionality reduction. Predicting behavior of an audience from this reduced but more reliable neural signal which reflects consensus of a group now becomes manageable with traditional machine learning techniques. Various aspects use additional information extracted from the stimulus itself or from viewer responses of a group of individuals to improve prediction of audience behavior.

Various aspects herein relate to predicting viewership or audience response from aggregated neural responses of a group of individuals. Viewership response or other behavioral responses of an audience to a particular media broadcast can be reliably inferred from the neural responses of a group of individuals experiencing that stimulus. Viewership or other audience behavioral response can include, for example, sample statistics such as audience or viewership size, retention, the number of postings on social networks, volume of related email traffic, purchasing behavior, voting behavior, educational exam outcomes, or any other form of aggregate group response. A media broadcast can be, for instance, a TV or radio program, a movie (or a scene thereof), a piece of music, or any other stimulus proceeding over time in a coherent or consistent fashion that is experienced by a large audience (individually or simultaneously). Various aspects described herein include collecting neural responses from a representative group of individuals, and, combined with historical data of viewership or audience behavioral response, establishing a predictor of audience response (e.g., viewership) to potential or real future broadcasts or other exposures to the media. These predictions can then be utilized to guide, e.g., broadcast programming, advertisement placement, advertising content, or content direction.

Various aspects predict audience behavioral response that may be of interest within or beyond the field of “neuromarketing”. A wide variety of behaviors can be of interest, e.g., viewership size for a motion picture of TV series, audience retention during commercials, the number of postings on one or more social network(s), “likes” on video clips in online social media, volume of tweets or email traffic in repose to a news broadcast, purchasing behavior in response to TV/movie/online advertising campaign, polling results following political TV advertising, test exam outcomes following the viewing of instructional videos, or any other form of aggregated behavior of a large audience in response to a video/audio stimulus.

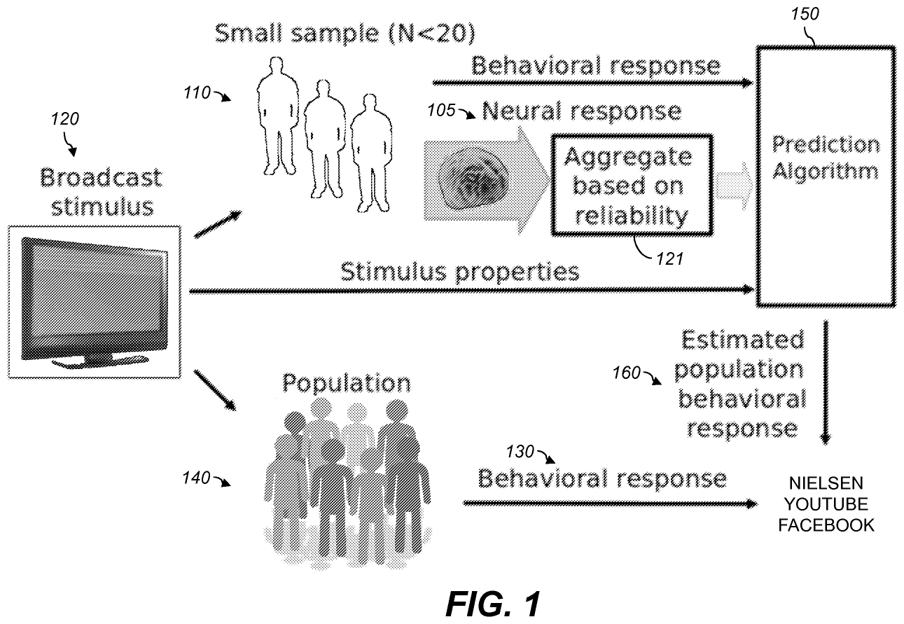

FIG. 1 shows a schematic representation of a prediction approach for predicting audience response from aggregated neural responses according to various aspects. The approach can involve:

a) Collecting neural responses 105 from a small sample of individuals 110 exposed to a stimulus 120;
b) Reducing the dimensionality or aggregating the neural responses into features or components based on within-subject reliability or across-subject agreement;
c) Using historical behavioral responses 130 on a larger audience 140 to train/learn the mapping from the aggregated data to the observed audience behaviors; and
d) Using this mapping to predict the behaviors on new stimuli from neural data of the sample.

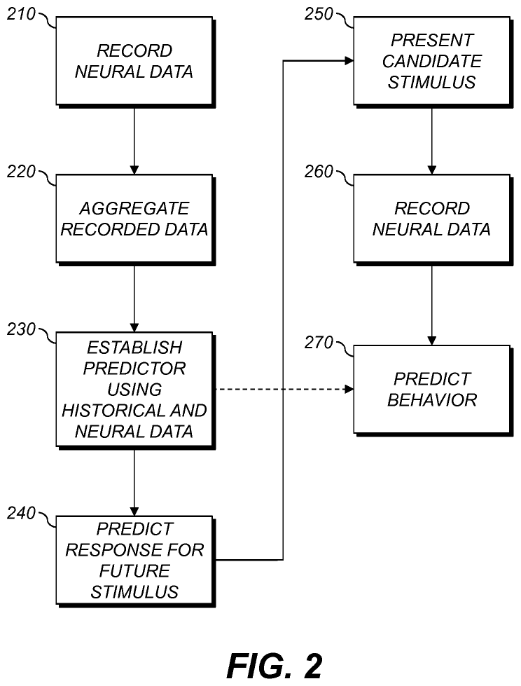

FIG. 2 shows a flowchart illustrating an exemplary method for collecting neural responses from a group of individuals to predict viewership or other audience behavioral responses. The steps can be performed in any order except when otherwise specified, or when data from an earlier step is used in a later step. The steps can be combined in various ways. In at least one example, processing begins with step 210. For clarity of explanation, reference is herein made to various components, groups, and data items shown in FIG. 1 that can carry out or participate in the steps of the exemplary method. It should be noted, however, that other components can be used; that is, exemplary method(s) shown in FIG. 2 are not limited to being carried out by the identified items.

In step 210, neural data 105 are recorded for a group 110 of individuals as they are presented with one or several media stimuli 120.

In step 220, the recorded data are aggregated to capture group statistics on neural response. See, e.g., step 121, FIG. 1.

In step 230, a predictor 150 of audience behavioral response is established based on historical data 130 using the aggregated neural data.

In step 240, this predictor is used to predict audience behavioral response 160 for future (potential) media exposures, by repeating steps 210 and 220 on a novel stimulus and using the predictor 150 of step 230 to generate a prediction 160 of the future audience response to the novel stimulus.

In various aspects, the group statistics of neural response 105 determined in step 220 indicate a reliability of neural response 105 to the media stimuli. Reliability can represent within-subject reproducibility or across-subject agreement and can include several independent measures of that reproducibility or agreement derived from a multitude of brain responses recorded with multiple sensors (e.g., EEG electrodes or fMRI voxels).

In various of these aspects, in step 220, measures of reliability are derived using correlated components analysis (CCA) or another signal analysis technique whereby neural signals are combined optimally such that correlation of neural responses across subjects or presentations is mathematically maximized. Further details of CCA are discussed below.

In various aspects, step 220 includes measuring reliability of neural responses. Reliability is computed as a correlation among combination(s) of neural signals such that reliability of the combined signals is maximal when the viewership or audience behavioral response of interest is maximal.

In various aspects, step 230 includes establishing the predictor so that, in addition to group statistics of neural responses, the predictor uses also available stimulus properties or behavior responses from the group.

### Neural Response Acquisition

Historical viewership or audience behavioral data 130 stemming from a previous broadcast or set of broadcasts is obtained, e.g., in or before step 230. Examples of such data include: estimates of the number of viewers for a given TV show on a particular day, or the number of viewers on a minute by minute basis of a particular TV broadcast, or the number of tweets related to a show on a given day, etc. In step 210, a stimulus for which viewership or audience behavioral responses are available is presented (potentially multiple times) to a relatively small sample, typically 10 to 50 individuals, appropriately selected to match the expected audience, or the audience of interest. During stimulus presentation, the individuals' neural activity is recorded through a neuroimaging modality such as electroencephalography (EEG), magnetoencephalography (MEG), or functional magnetic resonance imaging (fMRI). The individuals do not necessarily view the stimulus together—recording can be done at different times or different locations for different individuals. For each subject, a multivariate time series, referred to herein as X, encompasses that subject's observed neural response to the stimulus of interest.

### Dimensionality Reduction and Sample Aggregation

The acquired data records X are potentially high-dimensional (due to the large number of sensors or voxels) and can contain data points on a fine temporal scale. On the other hand, viewership or audience behavioral statistics are often univariate and acquired on a resolution in the order of a minute or higher. Thus, step 220 can include reducing both the dimensionality and temporal resolution of the acquired neural data in order to reduce the order of the forthcoming predictive model. The dimensionality reduction can be achieved by employing one of a number of techniques: principal components analysis, independent components analysis, or correlated components analysis (CCA). Reducing the temporal resolution can be achieved by sub-sampling the signals or binning the data into windows whose value depends in some functional form (for example, the mean, median, range, or any other statistic) on the finer sampled data in the bin. Performing dimensionality reduction and temporal downscaling yields a compact representation of the neural influence of the stimulus on each individual.

In order to construct the input to the prediction engine, a form of data aggregation which combines the data from multiple subjects into a sample-wide measure of the neural response to the stimulus is performed. This aggregation can take a number of forms, for example, computing the mean across all individuals, or the range or variance of responses across individuals, or computing a measure of reproducibility or reliability of the neural response across individuals (e.g., CCA, as described below), to summarize: mean, range, standard deviation, correlation, or any other group statistic of the neural response reliability resolved in time. Reliability can capture how reproducible neural responses are for a given subject under repeated exposures to the same stimulus. Alternatively, reliability can also represent how similar neural responses are between subjects exposed to the stimulus; this is referred to herein as the agreement of neural responses. The end result is an aggregated multivariate time series Y which captures neural response reliability and which can be utilized by the predictive model 150 in step 240 to generate estimates of the viewership or audience behavioral response. Other techniques that can be used to extract reliable features of the data include canonical correlation analysis, de-noising source separation, and hyper-alignment.

In an example of this technique, historical viewership or audience behavioral data stemming from a broadcast of a popular TV series premiere (AMC's “The Walking Dead”), including the intervening advertising segments, were analyzed. The viewership or audience behavioral data consisted of NIELSEN ratings on a minute-by-minute basis, as well as counts of Twitter posts referring to specific scenes of the show (positive, neutral and negative posts that could be associated with specific scenes). The neural response included recordings of EEG signals from 15 subjects, each sampled at 512 Hz and recorded at 64 electrodes corresponding to the standard locations of the 10/10 electrode placement system.

The data was spatially filtered across electrodes and subsequently correlated across subjects using CCA, described below, leading to 3 components which provided numerical values for neural response reliability on a minute-by-minute basis. The minute-by-minute features were then used to directly predict NIELSEN ratings, their temporal derivative (a measure of viewership or audience retention), and the number of “tweets” per scene.

### Correlated Components Analysis (CCA)

“Correlated components analysis” is a novel data analysis technique. It can identify spatial projections of high dimensional EEG data which maximize the temporal correlation between pairs of recordings. Specifically, let x₁(t) and x₂(t) be the multivariate time series recorded from two individuals (or repeated measures from the same subject): the aim is then to find a vector w such that the projection z₁(t)=w^{T}x₁(t) has maximal correlation with z₂(t)=w^{T}x₂(t). This can be achieved by maximizing

w*=argmax_{w}w^{T}(R₁₂+R₂₁)w/w^{T}(R₁₁+R₂₂)w (1)

where Rᵢⱼ=sumₜ xᵢ(t)xⱼ^{T}(t) are the spatial (cross-)covariance matrices of the recordings. This solution to this optimization problem is given by the following eigenvalue equation:

(R₁₂+R₂₁)⁻¹(R₁₂+R₂₁)W=D W (2)

where diagonal matrix D contains the eigenvalues of (R₁₂+R₂₁)⁻¹(R₁₂+R₂₁). More details on this technique can be found below. In particular, the estimates of Rᵢⱼ are preferably regularized as described below.

The dimensionality of the data has been reduced significantly from 64 or 128 channels (typical numbers of sensors in EEG/MEG) to just 2 or 3, by extracting 2 or 3 of the strongest correlated components. Furthermore, by calculating the correlation of these signal components in periods of a few seconds, the temporal resolution of the resulting reliability measure has been reduced from the millisecond range (typical sampling rate of EEG/MEG) to seconds. Both temporal and spatial reductions in dimensionality are useful and do not require information on viewership or audience response. Without such a reduction, efforts to train a predictor of viewership or audience response are bound to fail due to the curse-of-dimensionality, i.e. the mapping is severely under-constrained and the data is exceedingly noisy (typical SNR in EEG is −20 to −30 dB). With this technique, not just one measure of the strength of correlation is obtained. Instead the signals are reduced to several uncorrelated components that capture successively smaller levels of correlation across two datasets. In contrast, some prior approaches measure reliability simply as the correlation averaged across sensors, or as a raw sensor-wise correlation for each sensor. This latter approach is suboptimal as there may not be a good correspondence of a given sensor across two brains. Averaging across sensors on the other hand generates a less effective representation of correlation. Correlated components provide several dimensions that capture independent (uncorrelated) aspects of the neural data. An analysis using data from the repeated exposure to the same stimulus in one subject provides components that capture within-subject reproducibility of neural responses. An analysis using data from separate individuals provides components that capture across-subject agreement of neural responses.

### Modulated Correlated Components

A variant of this method captures reliability (correlation) across individual brain responses and provides high correlation at times of high viewership or audience response and low correlation at moments of low viewership or audience response. This variant is given by the following optimization problem:

w*=arginax_{w}w^{T}(H₁₂+H₂₁)w/w^{T}(L₁₂+L₂₁)w (3)

where Hᵢⱼ and Lᵢⱼ are the cross-covariance matrices of the recorded signals but computed separately during times of high and low viewership or audience response, respectively. The optimal spatial projection w again follows an eigenvalue equation:

(H₁₂+H₂₁)⁻¹(L₁₂+L₂₁)W=D W (4)

In this example, both the high and low eigenvalues provide useful discriminative spatial projections detecting moments of high and low correlation respectively. Thus, the components extracted here are modulated in their strength of correlation by the viewership or audience behavioral response. Both high and low correlated components can be used to predict viewership or audience behavioral response. This is similar to the approach that is used by the common-spatial-pattern (CSP) technique widely employed to train Brain Computer Interfaces. Audience behavioral response (e.g., viewership) has been used to perform dimensionality reduction. However, the algorithm has largely been trained on the correlation across many samples. Over-fitting is preferably avoided, e.g., by regularization and cross-validation, but the probability of overtraining is significantly reduced as compared to prior machine learning approaches to predict audience behavioral response (e.g., viewership) from the raw data.

### Robustness

The eigenvalue equations above are sensitive to noise and outliers. Care is preferably taken when estimating the relevant covariance matrices. Techniques that can be used for this are outlier rejection, shrinkage, and subspace reduction using principal component analysis.

### Correlations of Band-Pass Powers

Techniques discussed above can be applied directly to the raw EEG signals (after appropriate conditioning, e.g., high-pass filtering to remove slow drifts, or outlier rejection). Such techniques can also be applied to the instantaneous log-amplitudes of band-passed signals in different relevant frequency bands. Band-passed amplitudes have been shown to correlate across repeated viewings of the same stimulus in electro-corticograms (ECoGs). Both phase-locked evoked components in the raw EEG and non-phase-locked induced components captured by band-passed powers can be used in combination to extract reliable features across subjects. These features can then be used for predicting audience behavioral response.

Reproducibility and Agreement—within and Across Subject Correlations

Various methods above can be used to extract features (linear combinations of the neural signals) such that two data-sets are maximally correlated. The two data-sets can represent repeated exposures of the same subject to a stimulus, or can represent data collected from different subjects. In the case of repeated exposure in the same subjects, these correlations capture the reliability or reproducibility of the neural responses. When the signals represent neural data collected from different individuals, these correlations capture the agreement of neural responses across a group on individuals. In the examples above reliability is used as the feature for prediction of behaviors. However, agreement can also be used to predict an audience's behavioral response.

### Learning the Relationship Between Neural Signals and Ratings

In various aspects, in step 230, the parameters of predictive model 150 are tuned in a training procedure that employs historical viewership or audience behavioral responses in conjunction with neural response reliability to the corresponding stimuli acquired from a group of individuals who had not previously viewed the stimuli. The multivariate time series Y is fed into a learning algorithm which computes a set of parameters W which optimally predict the (known) ground-truth viewership or audience behavioral responses z. Here, “optimality” is used in a mathematical sense and can refer to any goodness-of-fit measure such as minimization of a least-squares error term or other suitably defined cost function. A multitude of learning algorithms can be used for this: for example, the least-mean-square algorithm, support vector machines, robust and sparse regression techniques, etc. Moreover, the model can take into account latent relations between neural responses and viewership or audience response; i.e., there is a temporal lag between neural “markers” and its manifestation in viewership or audience response. The model parameterized by W takes Y as an input and generates a prediction of the viewership or audience behavior which approximates the Ground-Truth Viewership or audience behavior in an optimal fashion.

### Subject Selection and Subset Selection

When collecting neural data from a group on individuals in step 210, the selection of subjects can be based on information about the target audience (e.g., age, gender, education, geographic location, or country of origin). After the data has been collected, the most predictive sample of individuals among the group can be selected. For instance, effective results have been obtained by selecting a subset of subjects based on the following criteria:

A. individuals with the best within-subject reliability in the data;
B. individuals with the “cleanest” data, e.g., the fewest number of outlier samples, or the lowest level of power-line noise;
C. agreement within a subset of the group: the subset of individuals that have the highest agreement with the group can be selected; or
D. behavioral response: individuals whose behavioral responses best agree with the large audience responses on historical data can be selected.
In general, after an initial set of subjects has been selected, any measure derived from the data or from the subjects' responses can be used to perform further subset selection.

### Predicting Viewership or Audience Behavior for Stimuli Prior to Broadcast

Referring to FIG. 2, having computed the optimal model parameters W, estimates can be generated of the audience behavior (e.g., viewership) in response to content that has not already been aired (step 240). In step 250, for each candidate stimulus or set of candidate stimuli, a group of subjects are presented with the stimulus and have their neural responses recorded (step 260) as described above. As with the training phase, this sample of individuals can be selected to match the target audience(s). The predictive model (with the parameters W obtained from training) then generates predictions of the viewership statistics or other audience behavior (step 270, using the model from step 230 as indicated by the dashed arrow).

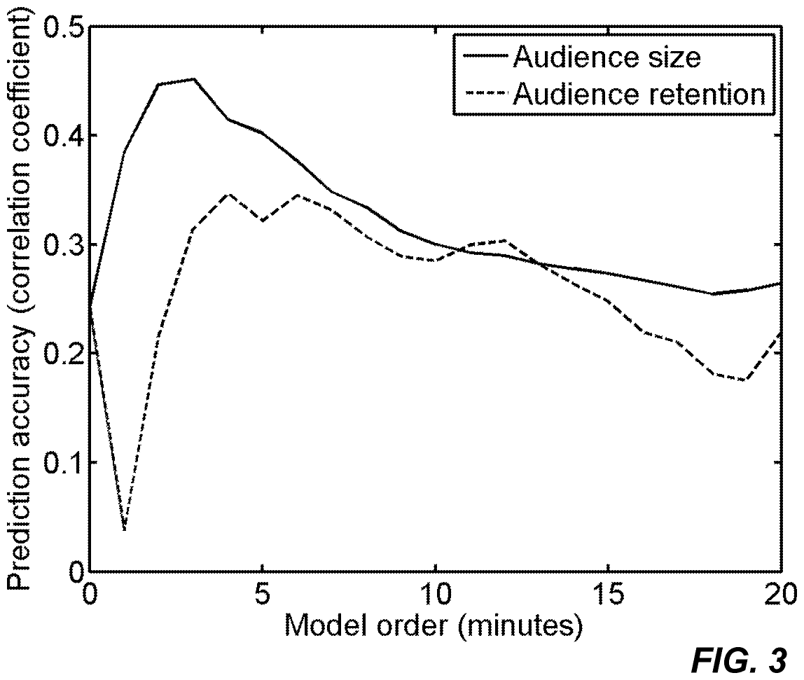

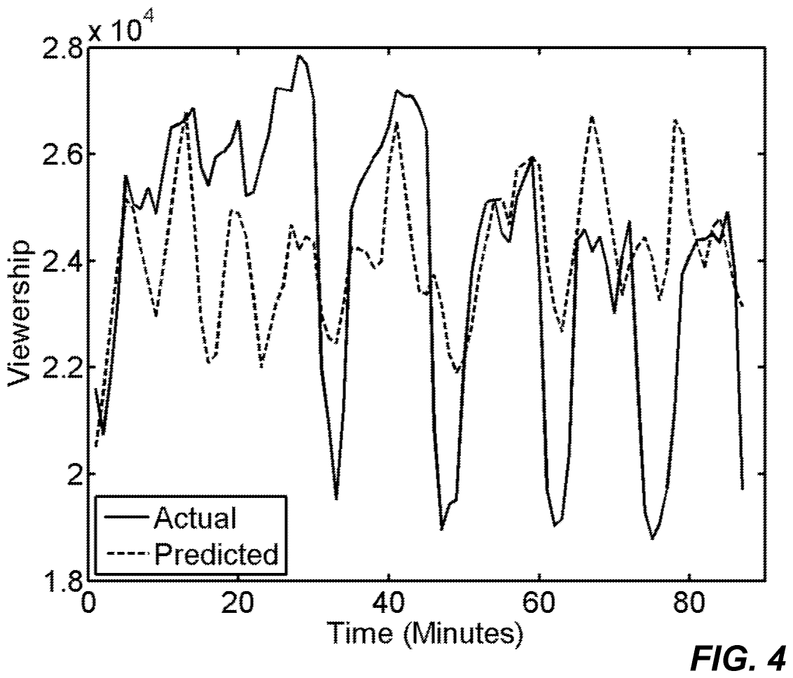

FIGS. 3 and 4 show an example of predicting the minute-by-minute NIELSEN ratings from the amount of neural response reliability (correlation across subjects) observed in a small sample of test subjects (N=15). Based on the amount of neural correlation observed in the past K minutes, where K is the model order, the model generates an estimate of the audience behavior (tune-in or viewership size) in the present minute. Optimal predictive performance, as measured here by leave-one-out cross-validation, is achieved by a filter which encompasses 3-4 minutes, depending on whether one is predicting the audience size (solid curve) or retention (dashed line).

In order to demonstrate the approach on the NIELSEN viewership-size data discussed above, a cross-validation procedure (involving partitioning the available data into “training” and “testing” data sets) has been used to predict the “unseen” viewership ratings across the episode. For each minute of the episode, the correlation in neural responses across our N=15 sample population was computed. A least-mean-square algorithm was then used to predict the viewership at minute m as a linear combination of the neural correlations at minutes m, m−1, . . . m−K, where K is the model order. The results are illustrated in FIGS. 3-4.

FIG. 3 illustrates that a model with a temporal aperture of 3-4 minutes effectively predicts the viewership size from neural correlation measures. Moreover, audience size is more predictable than audience retention (at least in this example).

FIG. 4 continues the example of FIG. 3. Dips in the ground-truth viewership size (solid line) correspond to the advertising segments, and occur in close correspondence with those predicted by the neutrally-informed model (dashed line). In general, the actual and predicted time series fluctuate in concert.

FIG. 4 depicts the time course of actual and predicted viewership size. It is readily apparent that “dips” in viewership occur in close correspondence to those predicted by the neural responses. In this example, these dips were determined to correspond to advertising segments. In general, the two curves tend to exhibit synchronous fluctuations showing a correlation of the prediction with the actual audience size of r=0.59.

### Additional Regression Variables

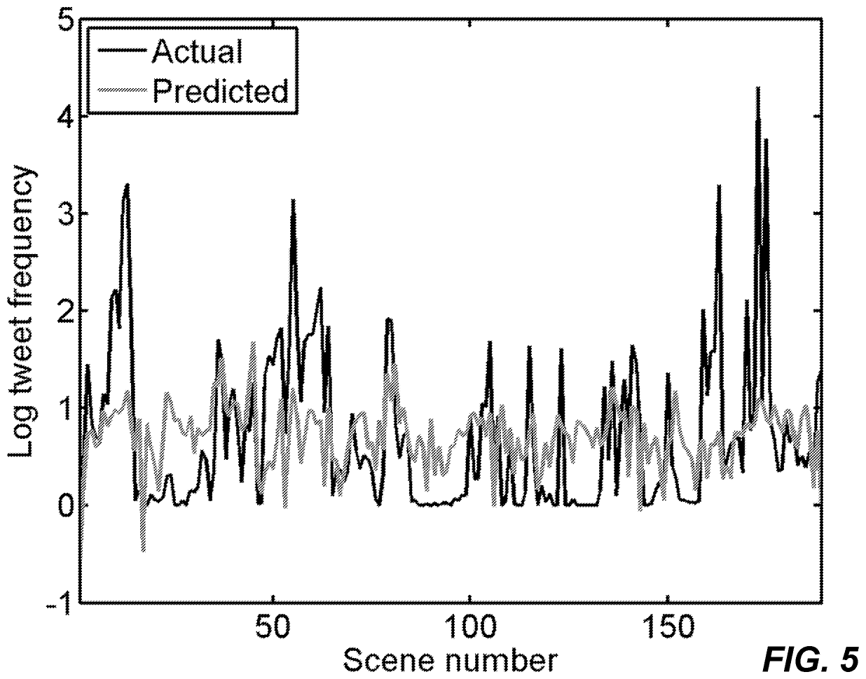

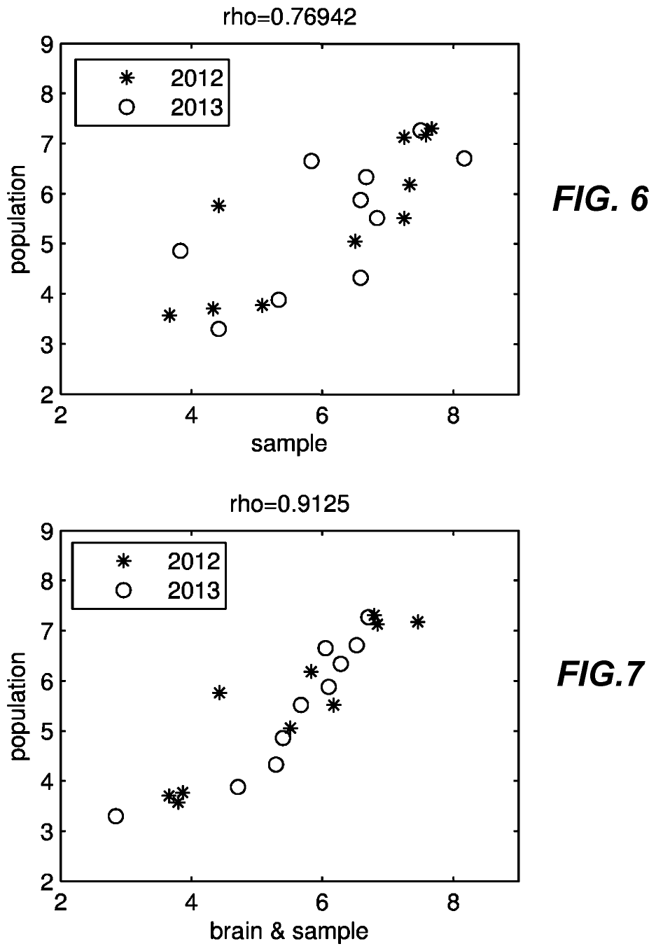

FIG. 5 shows an example of predicting “tweets,” short text messages from individuals broadcast to friends and to the public via the TWITTER microblogging Web site. In addition to the aggregated (reduced-dimensional) neural data, additional variables are used to predict audience behavior in this example. The regressors included the scene length in addition to neural data; training on the historical data indicated that longer scenes elicit higher tweet rates. Adding scene length and predicting log-tweet rate instead of raw number of tweets improved prediction performance from r=0.16 to r=0.37. Other variables can obviously be included into the prediction. For instance, when predicting subjective ratings of a program one can collect ratings also from the sample group and include these into the predictor of the larger audience for improved performance (see example in FIGS. 6-9). In general one can include all properties of the stimulus or behavioral responses from the small sample as regression variables to train a predictor.

FIG. 5 shows data of an experiment predicting the number of tweets per unit time (audience behavioral response, e.g., viewers' responses) elicited by each scene of the pilot episode of “The Walking Dead” from the neural reliability measured in a pool of test subjects. The two curves exhibit a significant correlation coefficient of 0.37. Thus, the reproducibility of the neural responses (from one subject to the next) is correlated to the amount of social response evoked by a certain scene.

As an example of viewership or audience behavioral response from the social media context, FIG. 5 depicts the results of predicting the number of tweets evoked by each scene of the pilot episode of “The Walking Dead.” For each scene, the reliability of the neural responses (in the space of the correlated components) during that scene was measured. A linear predictor was then fit to these neural reliability measures as well as scene length such that the resulting prediction best approximates the actual number of tweets per unit time elicited by the scene. The correlation between the predicted and actual number of tweets is substantial (r=0.37). This signifies that the neural reliability measured in a small sample of representative individuals carries information as to the social response of the larger audience.

FIGS. 6-9 show another example, the prediction of audience behavioral response to different video content, specifically to commercial advertising. Neural data on a small sample of individuals (N=12) on two sets of ads (10 ads aired during each of the 2012 and 2013 SUPER BOWL games) was collected. The USA TODAY Ad Meter ratings, which include subjective ratings of the ads collected from a large number of individuals with an on-line poll via the social media website FACEBOOK, were estimated. FIGS. 6-9 show that there is a strong correlation between the brain-based predictions and actual population ratings. These figures also demonstrate that the subjective ratings provided by the sample of individuals can be used to improve the prediction by including them in the learning step.

FIGS. 6-9 show an example of the prediction of subjective ratings for 10 SUPER BOWL commercials from 2012 and 2013 using aggregated neural signals. The respective correlation coefficient (“rho”) of observed and predicted ratings is shown over each graph in FIGS. 6-9.

FIG. 6 shows ratings of the population (USA TODAY Ad Meter ratings) versus average ratings provided by a small sample of individuals (N=12).

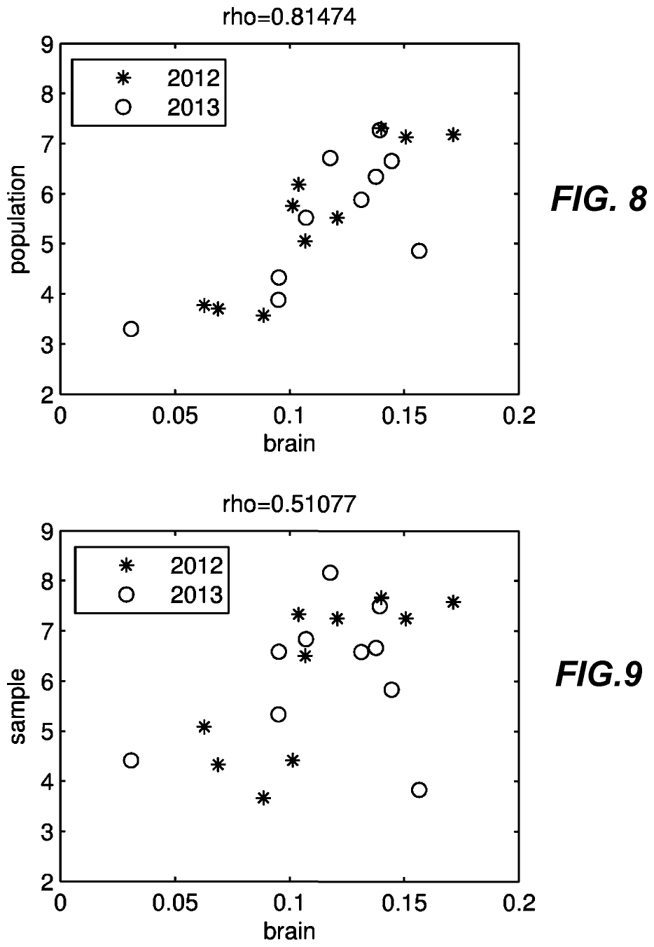

FIG. 8 shows prediction of the population ratings from the aggregated neural signals recorded from the brains of the individuals in the sample while watching the videos.

FIG. 7 shows prediction using a linear combination of aggregated brain signals and ratings of the sample group (vertical axis).

FIG. 9 shows prediction of the ratings of the sample using the corresponding aggregated brain signals.

Examples herein demonstrate this technique for US-wide NIELSEN ratings (number of viewers) on a minute-by-minute basis, and for the number of tweets associated with different scenes of a given TV program. Reliable prediction of USA Today Ad Meter ratings has also been demonstrated; those ratings reflect the responses of thousands of viewers across the US and beyond. These techniques can be used for predicting NIELSEN ratings among different populations (age, gender, ethnic groups, etc), or for predicting ratings across different programs (as with the rating of commercials discussed above with reference to FIGS. 6-9). These techniques can also be used to predict purchasing behavior in response to advertising, approval ratings in response to broadcast speeches, student performance in exams following viewing of video lectures, or other behavioral responses. The examples herein use EEG recordings as the neurological data, but the neural responses could include any functional imaging modality such as MEG, fMRI, fNIR, ECoG, PET or any other technique. In addition to neural responses, one can envision using physiological responses such as heart-rate, blood pressure, eye-movements (direction, velocity, number), etc. Reliability or reproducibility of these responses is determined across a group of individuals, and then the reliability measures are used as features with which to train a predictor of viewership or audience behavioral response.

Prior neuromarketing schemes and related techniques have relied on one of the following two designs.

Design 1, from what is known about functional neuroanatomy, determines the brain structure in which altered activity indicates the desired behavioral response. Examples of such structures are the nucleus accumbens (linked to product preference) or the orbitofrontal cortex (linked to willingness to pay). Then, present the stimulus-of-interest and “read-out” the level of activity in that fixed region (typically via BOLD responses measured using fMRI) as a proxy for the desired behavior.

Design 2, from what is known about neural oscillations, determines the frequency band and scalp location of the oscillations that are linked to a specific behavior. Examples are left-frontal theta band (4-8 Hz) oscillations that are linked to formation of long-term memories of presented advertisements, as well as left-right prefrontal cortex asymmetry, which indicates motivational valence. While presenting the stimulus-of-interest, the chosen frequency spectrum is computed via spectral analysis of MEG or EEG recordings, and again, the power, phase or spatial distribution (left-right lateralization) of the measured spectrum is used to index the desired behavior. Other methods rely on stimulus evoked responses characterized by their latency and polarity to the stimulus (in particular late components such as P300, N400, etc involving higher level cognitive processing). Changes in amplitude, spatial distribution, or timing can be indicators of certain properties of the stimulus.

The problem with these designs is that they strongly rely on the link between neural structure and function, which is still evolving in the neuroscientific literature. As a result, the read-outs from neuromarketing experiments may not necessarily correspond to the intended behaviors. Various aspects herein make no such functional assumptions, and instead employ a data-driven measure of neural reproducibility as the link between neural activity and subsequent behavior. Instead of a priori information about which brain regions or neural oscillations are indicative of the desired response, various algorithms herein automatically pull out signal components that are maximally correlated across the population and thus correspond to neural processing of the stimulus (as opposed to ongoing neural activity not related to the stimulus).

The approach taken here is also novel in that behavior of an audience is predicted not from the brain signals themselves, but rather, from a measure of their reliability or agreement across a group of individuals. This initial step of data reduction (raw signal into reliability/agreement) circumvents the “curse of dimensionality” that many learning or pattern recognition approaches would suffer from when trying to identify a predictive mapping approach from neural signal to behavior. In addition, by incorporating a learning step that combines several (uncorrelated) components of this neural reliability/agreement measure, one can potentially identify different mappings for a wide class of behaviors that are not limited to how engaging, effective or memorable a stimulus is.

These signal components are not restricted to originate from any specific brain areas, nor are they required to possess certain spectral, temporal or spatial properties. By computing a time-resolved measure of the level of correlation observed across the population in these components, a time series (time as the independent variable, correlation coefficient as the dependent variable) is obtained which quantifies the neural reproducibility elicited by the presented stimulus. Note that this is not the same as measuring the amount of neural activity across time, as is commonly the case with neuromarketing efforts. Prior schemes assume that high levels of activity correspond to a strong desired response; various aspects herein do not use that assumption. The proposed characterization of the neural reliability stemming from the stimulus constitutes a “post-design” offering. That is, for a given stimulus, the time-varying neural reliability quantifies the response of the experiment participants. This reliability time series can be used to infer the overall population response by feeding the reliability values into a prediction algorithm as described herein. This predictive model is fit from historical data from past stimuli—as such, our approach addresses the big question in neuromarketing, namely, whether neural measurements truly correspond to future consumption. Herein, models are designed to mathematically optimize the match between neural responses and future consumption, and then the models are used to make predictions about consumption of unreleased products or services. More specifically, the reliability measure can be optimized to be maximally predictive of the desired viewership or audience behavioral response as described above with reference to “Modulated correlated components”.

The general idea of using preexisting marketing communications to train algorithms which can then forecast outcomes of a new commercial campaign has been suggested. However, these schemes use levels of activation in the BOLD response, acquired via fMRI, as their features. These features are acquired on an individual subject basis. It should also be noted that while the idea of learning a predictive model from preexisting stimuli is mentioned, no examples of such analyses are provided.

Some prior schemes use a reliability measure to assess how engaging, effective or memorable a given stimulus is, i.e., they use neural signals to assess a property of the stimulus. In contrast, inventive aspects described herein use reproducibility as a basis for predicting an arbitrary future behavior of an audience (e.g., response to a scene in a movie or a commercial) via a learning algorithm, which may or may not be associated with those specific stimulus properties. In addition and in contrast to prior schemes, the prediction approach can also incorporate additional information from the focus group or the stimulus itself. By measuring the reliability of a stimulus for which data on subsequent population response is known, the relationship between the neural test-population reliability/agreement and subsequent overall behavioral population response is learned. Then, for novel stimuli, the reliability of the sample population's neural signals is used to generate predictions of the future (unknown) viewership or audience behavioral response. In contrast to prior art, reliability and agreement here are captured by several uncorrelated components of the neural signals which exhibit high or maximal correlation across subjects. Thus, this representation of reliability/agreement is multi-dimensional. This multi-dimensionality permits the prediction of a diversity of behaviors. At the same time, this reduced representation overcomes the ill-posed problem of mapping from a very high dimensional and noisy signal (brain activity) to behavior, an age-old and unsolved problem despite decades of research in neuroscience.

Various aspects use correlated components of ongoing EEG. These components can point to emotionally-laden attention and serve as a possible marker of engagement. Various aspects relate to electroencephalography, brain decoding, engagement, or naturalistic stimulation.

Recent evidence from functional magnetic resonance imaging suggests that cortical hemodynamic responses coincide in different subjects experiencing a common naturalistic stimulus. As described herein, neural responses in the electroencephalogram (EEG) evoked by multiple presentations of short film clips are used to index brain states marked by high levels of correlation within and across subjects. A novel signal decomposition method is formulated; this method extracts maximally correlated signal components from multiple EEG records. The resulting components capture correlations down to a one-second time resolution, thus revealing that peak correlations of neural activity across viewings can occur in remarkable correspondence with arousing moments of the film. Moreover, a significant reduction in neural correlation occurs upon a second viewing of the film or when the narrative is disrupted by presenting its scenes scrambled in time. Oscillatory brain activity is probed during periods of heightened correlation, and during such times there is observed a significant increase in the theta-band for a frontal component and reductions in the alpha and beta frequency bands for parietal and occipital components. Low-resolution EEG tomography of these components suggests that the correlated neural activity is consistent with sources in the cingulate and orbitofrontal cortices. Put together, these results suggest that the observed synchrony reflects attention- and emotion-modulated cortical processing which may be decoded with high temporal resolution by extracting maximally correlated components of neural activity.

The ability to reliably decode brain state from recordings of neural activity represents a major neuro scientific frontier. Up until recently, the majority of brain decoding research has employed an event-related design in which neural activity is regressed onto discrete event variables, allowing one to compute the neural correlates of a pre-defined and presumably fixed brain state. In natural settings, however, brain states are both continuous and transient. Moreover, the events eliciting state changes do not generally occur in a temporally regularized manner. Thus, there exists a need to track and index ongoing changes in cognitive state. In the absence of event markers, one possible solution is to regress the neural activity of one subject onto that of another, thus utilizing the correlation between multiple records to inform the state variables. Indeed, recent studies employing functional magnetic resonance imaging (fMRI) have revealed strong voxel-wise inter-subject correlations (ISC) across participants exposed to a common naturalistic stimulus (i.e., movie clips). Unfortunately, voxel-wise correlations in the blood oxygenation level dependent (BOLD) signal are unable to capture weaker activity that is distributed over distant cortical areas. Furthermore, the limited temporal resolution of fMRI constrains the potential of so-called “reverse-correlation” procedures that identify stimulus features eliciting the observed peaks in correlation. In other words, while fMRI may tell us f neural activity significantly correlates in response to a common stimulus, it will likely not be able to tell us precisely when this synchronization occurs. Finally, the hemodynamic response measured in fMRI only indirectly captures neural activity and does not allow for analysis of fast oscillatory activity (although it does correlate with oscillatory activity in the gamma band).

Various aspects overcome some or all of those deficiencies. Electroencephalography (EEG) can be used and offers a temporally-fine and direct measure of neural activity. EEG data are recorded during multiple views of short film clips and the temporal correlation of neural activity between the multiple views is measured. Instead of correlating raw signals in an electrode-to-electrode fashion, a signal decomposition method is employed to find linear components of the data with maximal mutual correlation. The resulting spatially filtered EEG can capture patterns of activity distributed over large cortical areas that would remain occluded in voxel-wise or electrode-wise analysis. Furthermore, the temporal resolution of EEG is sufficiently fine to capture rapid variations in amplitude and instantaneous power of ongoing neural oscillations. Patterns of neural oscillation have long been associated with cognitive functions such as attention (alpha-band activity), emotional involvement (beta oscillations) and memory encoding (theta activity). Thus, utilizing EEG permits relating the measured correlations to ongoing oscillatory activity, which can be representative of the cognitive states involved during synchronized periods.

The measure of correlation presented here is fundamentally different from prior schemes that only capture coincidence of high or low activity in the hemodynamic response. Here, the high temporal resolution of EEG is used to measure correlation in time between two viewings. Hence, the spatial components extracted here capture not only coincidence, but rather, they represent neural activity that similarly tracks or follows the stimulus. This measure is employed to investigate the link between neural correlation and viewer “engagement”—a cognitive state which lacks a rigorous definition in the neuroscience context and which is defined herein as “emotionally-laden attention.” In addition to the scientific value afforded by uncovering the neural substrates of engagement, the ability to monitor engagement in an individual or population has potential application in several contexts: neuromarketing, quantitative assessment of entertainment, measuring the impact of narrative discourse, and the study of attention-deficit disorders. The statistically optimized measure of brain synchrony described herein can closely correspond to the level of engagement of the subject during viewing. In order to demonstrate this, the expected level of engagement can be manipulated in various ways. The measure of neural correlation has been determined to act as a regularized and time-resolved marker of engagement. Specifically, analysis reveals that peaks in this neural correlation measure occur in high correspondence with arousing moments of the film, and fail to arise in amateur footage of everyday life. Moreover, when the presentation of the film clip is repeated, or when it is shown with its scenes scrambled in time, a significant decrease in correlation is observed. Additionally, the instantaneous power in conventionally-analyzed EEG frequency band is probed. Significant co-variation of the activity in these bands with the optimized correlation measure has been demonstrated. While parietal and occipital power in the alpha and beta bands are decreased during peaks in synchrony, frontal theta power is increased during time windows of heightened correlation. Finally, low-resolution source localization analysis suggests that the components of correlated scalp activity are consistent with sources in the cingulate and orbitofrontal cortices. These results suggest that modulation of cortical processing during attention- and emotion-laden states leads to the observed between-view correlation, and such moments of “engagement” may be decoded from the EEG down to a one second time resolution.

### Materials and Methods

### Extraction of Maximally Correlated Components

Herein is described an analysis technique that is suitable for the continuous stream of neural activity generated during viewing of these film clips. With natural stimuli such as video, there may not be well-defined epochs that could be used with traditional methods of analyzing evoked or induced responses in EEG. Thus, instead of regressing the EEG signal against predefined discrete moments in time, the signal is correlated with the data from a second viewing that serves as a time-accurate reference for analysis. The second viewing can be by the same or a different subject. Electrodes can be combined linearly so as to identify, if necessary, distributed sources of neural activity instead of relying on individual voltage readings on the scalp. The traditional technique for extracting linear combinations of data with maximal correlation is canonical correlation analysis. Unfortunately, canonical correlation analysis requires the canonical projection vectors (i.e. spatial filters) to be orthogonal. This is not a meaningful constraint as spatial distributions are determined by anatomy and the location of current sources and are thus not expected to be orthogonal. Moreover, canonical correlation analysis assumes that each of the two data sets requires a different linear combination, thus doubling the number of free parameters and unnecessarily reducing estimation accuracy. By dropping this assumption—a sensible choice as the two data sets are in principle no different—fewer degrees of freedom are present. This permits removing the constraint on orthogonality. The resulting algorithm, which maximizes the Pearson Product Moment Correlation Coefficient and is referred to herein as “correlated components analysis,” includes simultaneously diagonalizing the pooled covariance and the cross-correlations of the two data sets. The linear components that achieve this can be obtained as the solutions of a generalized eigenvalue equation (eq. (7)), as can other source separation algorithms used in EEG.

### Correlated Components Analysis

Details of a component analysis technique which has been specifically designed to find linear components of the data that are maximally correlated in time when comparing two different renditions are now provided.

Given two data sets X₁∈ [symbol] ^{D×T} and X₂∈ [symbol] ^{D×T}, where D is the number of channels (i.e., electrodes) and T the number of time samples, it is desirable to find a weight vector w∈ [symbol] ^{D} such that the resulting linear projections y₁=X₁^{T}w and y₂=X₂^{T}w exhibit maximal correlation. For example, X₁ and X₂ may be the EEG data records stemming from two viewings of the movie clip. Moreover, w is a spatial filter which linearly combines the electrodes such that the resulting filter outputs y₁ and y₂ recover correlated sources. Formally, the optimization problem seeks to maximize the Pearson Product Moment Correlation Coefficient between y₁ and y₂ (assuming zero-mean data):

$$\begin{matrix}\hat{w} = arg {max}_{w} \frac{{y}_{1}^{T} {y}_{2}}{‖ {y}_{1} ‖ ‖ {y}_{2} ‖} \\ arg {max}_{w} \frac{{w}^{T} {R}_{12} w}{\sqrt{{w}^{T} {R}_{11} w} \sqrt{{w}^{T} {R}_{22} w}}\end{matrix} , \qquad ( 5 )$$

where the sample covariance matrices are denoted by

$${R}_{j} = \frac{1}{T} {X}_{i} {X}_{j}^{T} , i , j ∈ \{ 1 , 2 \} .$$

Differentiating [5] with respect to w and setting, to zero yields;

$$\frac{{σ}_{11} {σ}_{22}}{{σ}_{12}} {R}_{12} w = ( {σ}_{22} {R}_{11} + {σ}_{11} {R}_{22} ) w , \qquad ( 6 )$$

where σᵢⱼ=w^{T}Rᵢⱼw denote scalar power terms required to bring the two data sets onto the same scale. While prior knowledge of σᵢⱼ is often not available, the assumption can be made that the two data sets have similar power levels, and thus σ₁₁≈σ₂₂. In various aspects, the power levels of recordings stemming from two viewings (or two subjects) are roughly equivalent. Moreover, the cross-covariance matrix R₁₂ is symmetrized to arrive at the following eigenvalue equation:

(R₁₁+R₂₂)⁻¹(R₁₂+R₂₁)w=λw, (7)

where

$$λ = \frac{{σ}_{12}}{{σ}_{11}} .$$

As [7] is a generalized eigenvalue problem, there are multiple (and not necessarily orthogonal) solutions. The weight vector that maximizes the correlation coefficient between y₁ and y₂ follows as the principal eigenvector of (R₁₁+R₂₂)⁻¹ (R₁₂−R₂₁), with the optimal value of the correlation given by the corresponding eigenvalue. Moreover, the second strongest correlation is obtained by projecting the data matrices onto the eigenvector corresponding to the second strongest eigenvalue, and so forth. As the decorrelation (correlation matrix inverse) operation is sensitive to dimensions dominated by noise, the algorithm is effectively regularized by truncating the eigenvalue spectrum of the pooled covariance to the K strongest principal components. The value of K serves as a regularization parameter: the larger the number of whitened components, the stronger the optimal correlation. However, lower values for K will shield the learning, algorithm from picking up spurious correlations from noisy recordings.

### Intra and Inter Subject Correlation (IASC, ISC)

The two data matrices X₁ and X₂ used to compute the correlation and cross-correlation matrices in the forthcoming results are defined here. For the first analysis, of within-subject correlations, the subject-aggregated data matrices are defined as follows:

$${\ddot{X}}_{1} = [ \begin{matrix}\begin{matrix}\begin{matrix}{X}_{1}^{( 1 )} & {X}_{1}^{( 2 )}\end{matrix} & ⋯\end{matrix} & {X}_{1}^{( N )} ]\end{matrix} \qquad ( 8 )$$

$${\ddot{X}}_{2} = [ \begin{matrix}\begin{matrix}\begin{matrix}{X}_{2}^{( 1 )} & {X}_{2}^{( 2 )}\end{matrix} & ⋯\end{matrix} & {X}_{2}^{( N )} ]\end{matrix} ,$$

where Xᵢ⁽ⁿ⁾, i∈{1, 2}, n={1, 2, . . . , N} the BEG data record from the ith viewing of the movie by the nth subject. For the analysis that is concerned with across-subject correlations, aggregated matrices \ddot{X}₁ and \ddot{X}₂ are defined such that the subsequent correlation considers all unique combination of pairs of subjects. For example, for a three-subject population:

$${\ddot{X}}_{1} = [ \begin{matrix}\begin{matrix}{X}_{1}^{( 1 )} & {X}_{1}^{( 1 )}\end{matrix} & {X}_{1}^{( 2 )} ]\end{matrix} \qquad ( 9 )$$

$${\ddot{X}}_{2} = [ \begin{matrix}\begin{matrix}{X}_{1}^{( 2 )} & {X}_{1}^{( 3 )}\end{matrix} & {X}_{1}^{( 3 )} ]\end{matrix} ,$$

where the above matrices correlate the records from viewing 1 only. Analogous definitions hold for the second viewing. As it is expected that only certain scenes evoke significant correlations, the correlations are computed in a time-resolved fashion by employing a sliding window with a 5 second duration with a shift of the window occurring every second (80% overlap between successive windows).

### Forward Model

Given a set of linear spatial filters W and the data covariance matrix R, the forward models A=RW(W^{T}RW)⁻¹ represent the scalp projections of the synchronized activity extracted by the projection vectors W.

### Source Localization

The standardized low resolution brain electromagnetic tomography package (sLORETA, version 20081104) is used to translate the obtained forward models into distributions of underlying cortical activity.

### Spectral Analysis

In order to compute the instantaneous power of EEG in the theta (4-8 Hz), alpha (8-13 Hz), and beta (13-30 Hz) frequency bands, a complex Morlet filter can be employed. This filter can be of the form

$$h ( t ) = {ae}^{2 π {if}_{c} t} {e}^{- {( \frac{t}{\sqrt{2} σ} )}^{2}}$$

with the following parameters for each band:

theta: α=0.05, f_{c}=6, σ=0.12, −0.5≤t≤0.5 s

alpha: α=0.05, f_{c}=10, σ=0.1, −0.33≤t≤0.33 s

beta: α=0.2, f_{c}=20, σ=0.075, −0.33≤t≤0.33 s

The instantaneous power follows as the squared magnitude of the complex filter output y(t)=h(t)*x(t), where * denotes the convolution operator.

### Experiment

A study was performed. A total of 20 subjects with self-reported normal or corrected-to-normal vision and normal hearing participated in the study. The minimum, median, and maximum age of the subjects was 21, 24, and 45, respectively, with 14 males and 6 females volunteering. All experiments were approved by the Institutional Review Board of the CITY COLLEGE OF NEW YORK and all subjects gave written informed consent prior to the experiment. Subjects were instructed to sit comfortably, attentively watch the forthcoming movie clips, and refrain as much as possible from overt movements. Each subject was then presented with three 6-minute movie clips, with each clip being shown twice. The order of the three clips was randomized across subjects, but the order was preserved within each subject (for example, a typical session included the order M2 M1 M3 M2 M1 M3). The movie clips chosen were from the following films: “Bang! You're Dead,” (1961) directed by Alfred Hitchcock as part of the Alfred Hitchcock Presents series; “The Good, the Bad, and the Ugly,” (1966) directed by Sergio Leone; and a control film which depicts a natural outdoor scene on a college campus.

### Data Collection and Pre-Processing

The EEG was recorded with a BioSemi Active Two system (BioSemi, Amsterdam, Netherlands) at a sampling frequency of 512 Hz. Subjects were fitted with a standard, 64-electrode cap following the international 10/10 system. In order to subsequently remove eye-movement artifacts, the electrooculogram (EOG) was also recorded with four auxiliary electrodes. All signal processing was performed offline in the MATLAB software (Mathworks, Natick, Mass.). After extracting the EEG/EOG segments corresponding to the duration of each movie, the signals were high-pass filtered (0.5 Hz) and notch filtered (60 Hz). Eye-movement related artifacts were removed by linearly regressing out the four EOG channels from all EEG channels. The regression approach was chosen over component-based techniques used by prior schemes. EEG samples whose squared magnitude falls above four standard deviations of the mean power of their respective channel were replaced with zeros. In this example, without regressing eye-movement related activity from the data, the forthcoming correlated components showed stereotypical signatures of eye movements, as expected given that well-edited films are known to evoke similar scan paths in viewers. After regression, these components disappeared.

### Statistical Significance

In order to establish significance of the time-resolved correlation, a permutation test approach is employed. To yield correlation values under the null hypothesis, the correlations with one of the two records (either from a second viewing or subject) scrambled in time are computed: the second record is divided into 5-second blocks, with the order of the blocks then randomly shuffled. All significance tests are corrected for multiple comparisons using the false discovery rate.

### Results

### Peaks in Intra-Subject Correlations (Iasc) Occur at Momentous Film Events

Intra-subject correlations (IaSC) between the two viewings and their relationship to stimulus characteristics are now described. To that end, subject-aggregated data matrices are constructed by concatenating in time the data from multiple subjects separately for each viewing (see eq. (8)). The aggregated data is substituted into the eigenvalue equation of eq. (7) to yield the optimal spatial filters and resulting components. For each of n=10 subjects, the coincidence in neural activity across the two viewings is then measured by computing the correlation coefficient in the component space. The population IaSC follows as the average of these correlation coefficients across all subjects.

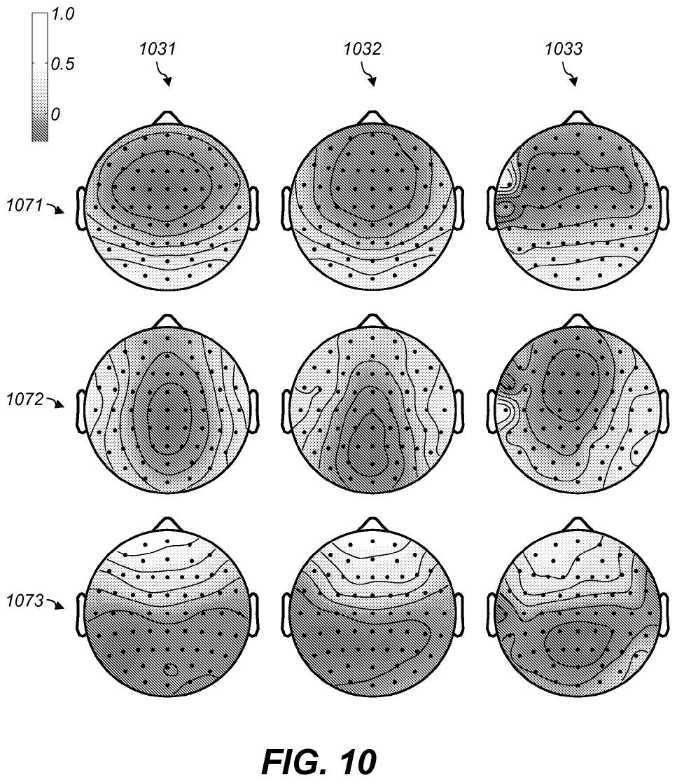

FIG. 10 depicts the top three correlation-maximizing components, shown in the form of “forward-models” (see “Methods,” below) which depict the projection of the correlated neural activity on the scalp. Lighter values indicate positive correlation of a source and an EEG sensor; darker values indicate negative correlation (this is described in Parra et al., “Recipes for the linear analysis of EEG,” Neurolmage 28 (2005) 326-341). FIG. 10 shows the spatial topographies of the correlated components observed during two critically-acclaimed films and one amateur control. The scalp projections of the first three maximally correlated components show appreciable congruence across the three films shown. Rows 1071, 1072, and 1073 represent the first, second, and third maximally correlated components, referred to herein as “C1,” “C2,” and “C3.” Column 1031 shows results for “Bang! You're Dead”, column 1032 shows results for “The Good, the Bad, and the Ugly,” and column 1033 shows results for the control film. Lighter shades represent positivity and darker shades represent negativity.

There is an appreciable level of agreement in the forward-models across the three movies shown, including the amateur film depicting an outdoor scene lacking noteworthy action. The first component (row 1071) is symmetric and marked by an occipital positivity and parietal negativity. The second component (row 1072) is also symmetric with positivity over the temporal lobes and negativity over the medial parietal cortex. Meanwhile, the third component (row 1073) shows a strong frontal positivity with broad temporal-parietal-occipital negativity.

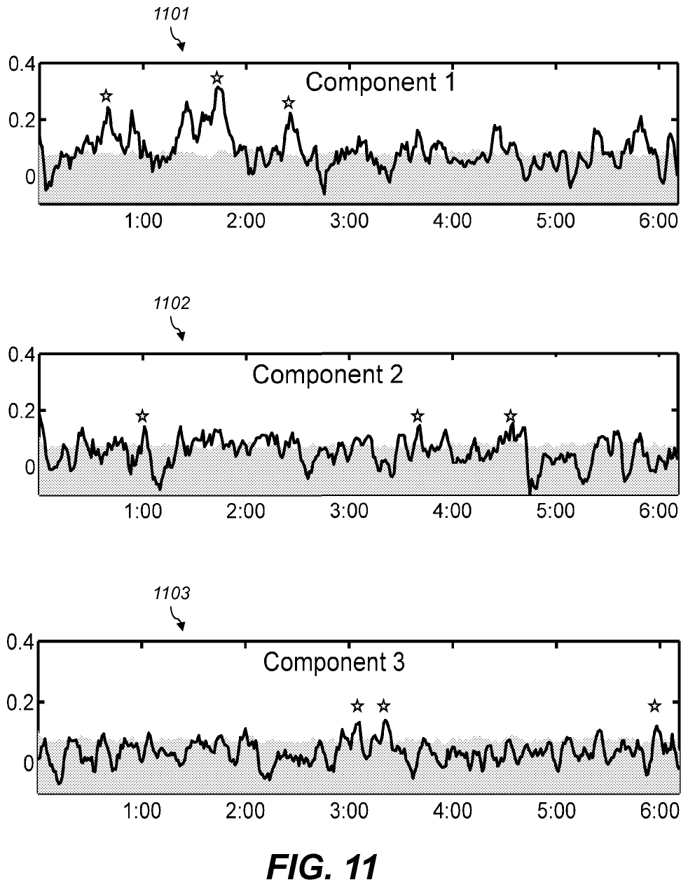

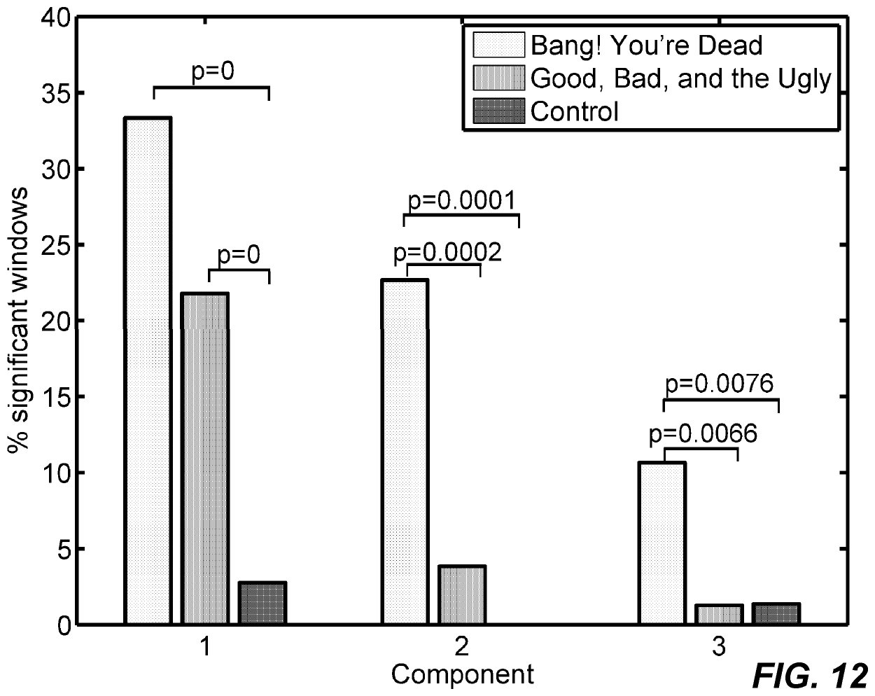

The resulting population correlation coefficients are shown as a function of movie time for “Bang! You're Dead” in FIG. 11. The grey shaded area indicates the correlation level required to achieve significance at the p<0.01 level (using a permutation test). The first component shows extended periods of statistical significance, staying above the significance level for approximately 33% (corrected for multiple comparisons by controlling the False Discovery Rate) of the film. More importantly, the peaks of the population IaSC correspond to moments in the clip marked by a high level of suspense, tension, or surprise, often involving close-ups of the young protagonist's revolver (which the audience, but not the boy, knows is genuine and contains one bullet) being triggered. Star icons mark examples of such moments. The correlation time series of the second component spends approximately 23% of the film duration above the significance level, with local maxima seeming to coincide with scenes of cinematic tension involving hands (i.e., the protagonist's Uncle realizes that his revolver is in the hands of the boy; the protagonist points the real gun at an approaching mailman; the boy finds a case of bullets in the guest room). Finally, the population IaSC as measured in the space of the third component is significant for approximately 10% of the clip duration, exhibiting peaks at moments roughly linked to anticipation. FIG. 12 summarizes the proportion of significantly correlated time windows of each component and movie. Components 1, 2, and 3 correspond respectively to rows 1071, 1072, 1073 (FIG. 10). EEG responses to the control film show little significant correlated activity. A standard hypothesis test of proportions was employed to test whether pairs of observed ratios are drawn from disparate distributions. Where significant, the corresponding p-values are indicated. In the first component, for example, there is a significant increase in the proportion of significantly correlated time windows in the two critically-acclaimed films as compared to the control film.

FIG. 11 shows the within-subject correlation over time for “Bang! You're Dead.” The within-subject correlation peaks at particularly arousing moments of this film, with over 30% of the film resulting in statistically significant correlations in the first component (FIG. 12). On the other hand, any extended periods of statistically significant correlation fail to arise during the control clip.

Population Iasc is Strongly Attenuated when “Meaning” of Stimulus is Lost

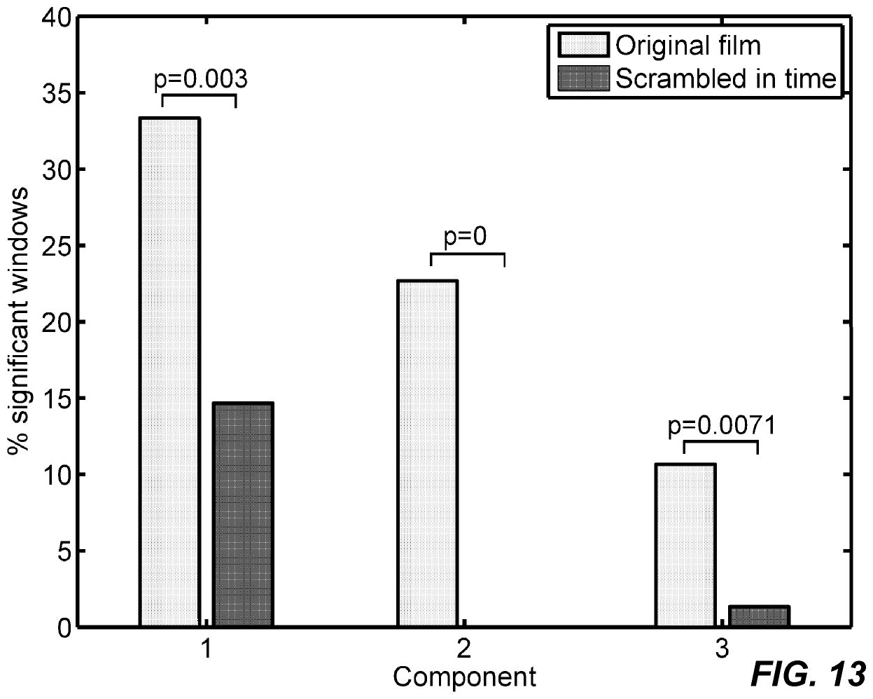

FIG. 13 shows data as in FIG. 12 for “Bang! You're Dead,” presented with its scenes scrambled in time. A significant reduction in neural correlation was observed. Specifically, a further control was constructed by extracting 46 scenes of “Bang! You're Dead”, randomly shuffling their temporal order, and recording the neural activity in response to this temporally reordered, but otherwise identical, stimulus (for this experiment, a separate group of n=10 subjects was employed, and each subject viewed the scrambled film twice). Comparing the neural responses of the scrambled film with the original version controls for the low-level visual and auditory features of the stimulus which are identical in both conditions. On the other hand, the meaning, affect, and suspense are presumably elevated when viewing the film clip in its original order. As shown in FIG. 13, the proportion of statistically significant windows is reduced to 14%, 0% (no significant time windows), and 1% for components 1, 2, and 3, respectively, in the scrambled film. Once again, a hypothesis test of proportions reveals that these reductions are statistically significant at the p<0.01 level.

### Inter-Subject Correlation (Isc) Decreases During Second Viewing

The effect of prior exposure to the stimulus on the resulting neural correlation was investigated. The population inter-subject correlation (ISC) was measured during the first and second viewings of the clips for n=10 subjects. Analogously to the measure of population IaSC defined above, aggregated matrices were constructed such that the subsequent correlation considers all unique combinations of pairs of subjects (see eq. (9)). Once these concatenated data sets are constructed, the eigenvalue problem of eq. (7) is solved to yield the spatial filters maximizing the ISC across the entire population.

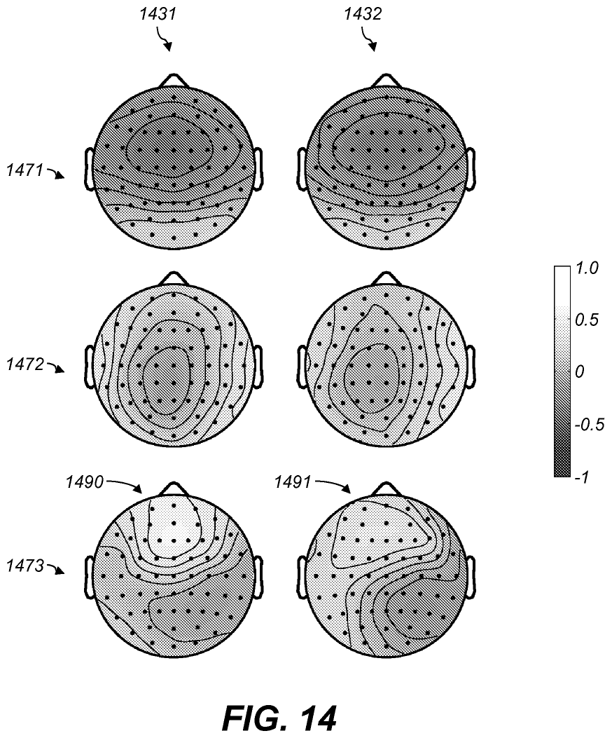

FIG. 14 depicts the scalp projections of the maximally-correlated (across-subject) components for “Bang! You're Dead.” Rows 1471, 1472, and 1473 correspond respectively to the first, second, and third such components, referred to as C1, C2, C3, respectively. The data in col. 1431 are similar to those maximizing the population IaSC as shown in FIG. 10, col. 1031. This is an intuitively satisfying result, as it stands to reason that the neural “sources” responsible for the correlated stimulus-driven activity across viewings of the same individual would also lead to across-subject reliability. While a high level of congruence exists between the forward models of the first and second viewings, shown in col. 1431 and col. 1432, respectively, the third component of the first viewing exhibits stronger frontal positivity (area 1490) as compared to the second viewing (area 1491).

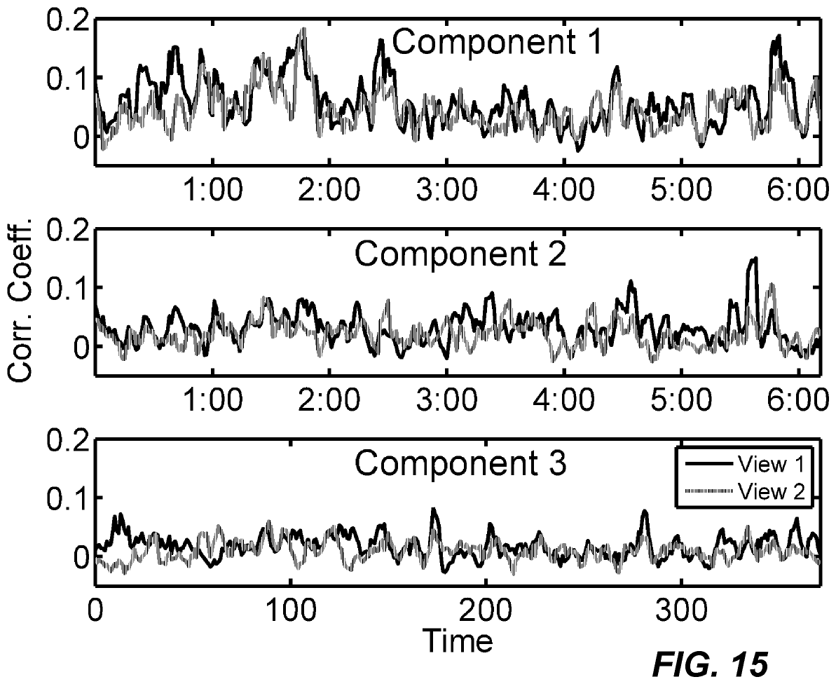

FIG. 15 depicts the time-resolved correlation coefficients averaged across subject pairs computed for each viewing. The Wilcoxon signed rank test was performed to determine the probability that the differences in population ISC between the two viewings could have originated from a zero-median distribution. For all three components, the null hypothesis was rejected (p=0.004, p=0.012, p=0.005, for components 1, 2, and 3 respectively)

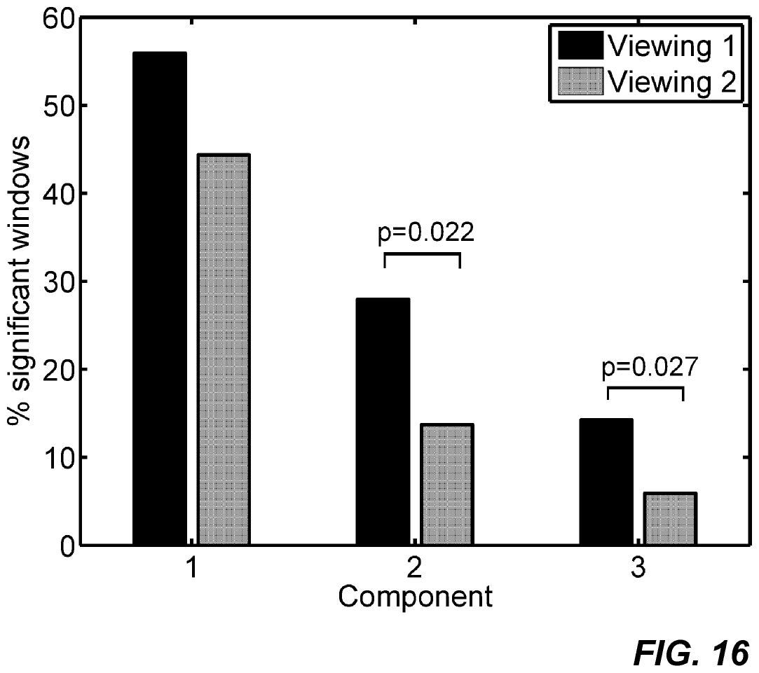

FIG. 16 shows a statistically significant reduction in the proportion of time windows showing significant correlation during the second viewing in the second (p=0.022) and third components (p=0.027).

FIGS. 14-16 show the effect of prior exposure on neural correlation. The scalp projections of the components maximizing population ISC during the first viewing are largely congruent to those stemming from viewing 2 (FIG. 14). However, the resulting time-resolved correlation measures are significantly lower during the second viewing (FIG. 15). Furthermore, more time windows exhibit statistically significant ISC in the first viewing (FIG. 16).

### High Neural Correlation Marked by Decreased Alpha and Increased Theta

Due to the fine temporal resolution inherent to EEG, it is possible to uncover the frequency bands that are systematically increased (or decreased) during periods of high correlation. For example, desynchronization in the alpha band has been shown to be associated with increased attention, while increased alpha-band oscillations presumably reflect an attention suppression mechanism. As a result, one may expect an inverse relationship between alpha power and decoded engagement.

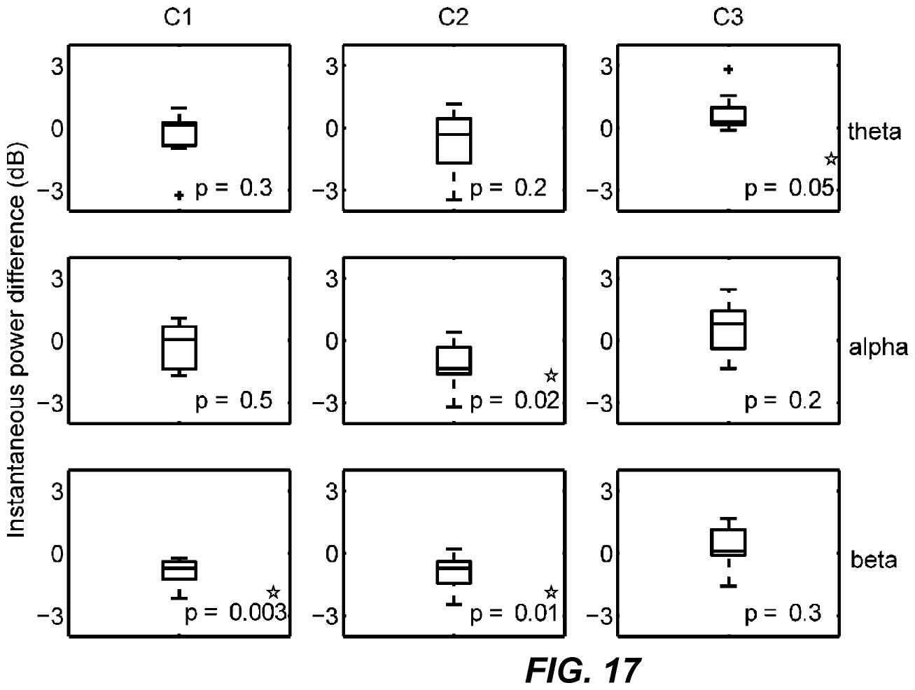

FIG. 17 shows results of a comparison of instantaneous power at several nominal EEG frequency bands (collapsed across subjects and viewings) during times of high within-subject correlation with that observed during low-correlation periods. For each subject, the mean instantaneous power during temporal windows in the top and bottom 20 percent of the population IaSC was computed, and then the power differences (high correlation versus low correlation, n=10) were tested for statistical significance using a one-sample Student's t-test. This procedure is performed in the component space: that is, the instantaneous powers are computed on the spatially filtered EEG. FIG. 17 displays the corresponding boxplots of differences in instantaneous power. Each boxplot displays the median (central mark), the 25 and 75 percentiles (box edges), extrema (whiskers), and samples considered outliers (“plus” signs). Columns C1, C2, and C3 correspond to the three maximally-correlated components, as described above. Rows “theta,” “alpha,” and “beta” correspond to those EEG frequency bands.

Effects deemed to be statistically significant are marked with star icons, and p-values are listed in each individual boxplot. As expected, there is a significant decrease in alpha power, measured in the space of the second (temporal-parietal) component, during periods of high IaSC. Moreover, the power in the theta band of the third (frontal) component is significantly increased during highly-correlated times—synchronization of frontal theta power with a concurrent decrease in alpha power has been linked to the encoding of new information. It has also been shown in an fMRI study that successful encoding of episodic memory is correlated with high ISC during initial exposure. Finally, a strong reduction in beta power in both the first and second components is shown—a decrease in temporal beta has been associated with so-called “intake” tasks, or those that require sustained monitoring of external emotionally-laden stimuli.

FIG. 17 shows differences in instantaneous power during moments of high versus low neural correlation. Distributions are constructed along the subject dimension (n=10, with statistically significant effects denoted with a star icon). High correlation windows are marked by synchronization of theta activity in the third component, desynchronization of alpha in the second component, and desynchronization of beta in the first and second components.

### Source Analysis Suggests Emotional Involvement

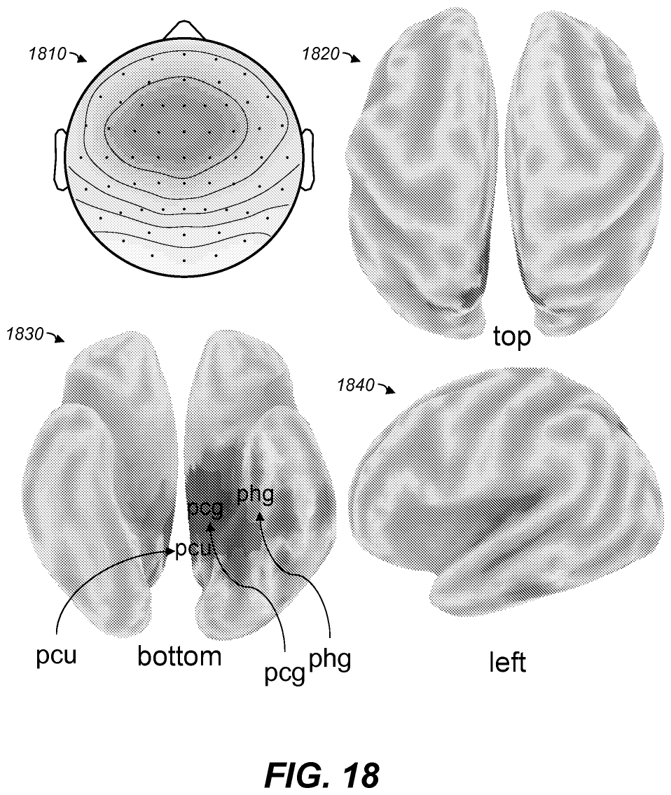

While the spatial resolution of EEG is inherently poor, low-resolution tomography (LORETA) of scalp potentials has been extensively employed to suggest possible cortical origins of the observed activity. Therefore, LORETA estimates were computed of the neural current source distributions explaining the scalp projections of the synchronized activity. The results are illustrated in FIGS. 18-20.

FIGS. 18-20 show sources of correlated neural activity for components 1, 2, and 3, respectively. The scalp projections 1810, 1910, 2010 of the correlated activity are shown in the top left of each pane; lighter shades indicate more positivity (closer to +1 on the scale of FIG. 14) and darker shades indicate more negativity (closer to −1 on the scale of FIG. 14). The estimated distributions of cortical sources are depicted in the remaining three panes: top views 1820, 1920, 2020; bottom views 1830, 1930, 2030; and left views 1840, 1940, 2040. Darker shading indicates a stronger activation or recruitment of the corresponding brain area. Anatomical locations shown are approximate.

Referring to FIG. 18, the correlated activity of component 1 suggests involvement of the posterior cingulate gyms (Brodmann Area 31, labeled pcg), the parahippocampal gyrus (Brodmann Area 27, phg), and precuneus (Brodmann Area 7, pcu). The postcentral gyrus (pocg) and paracentral lobule (pacl) are implicated in the localization of the activity in component two.

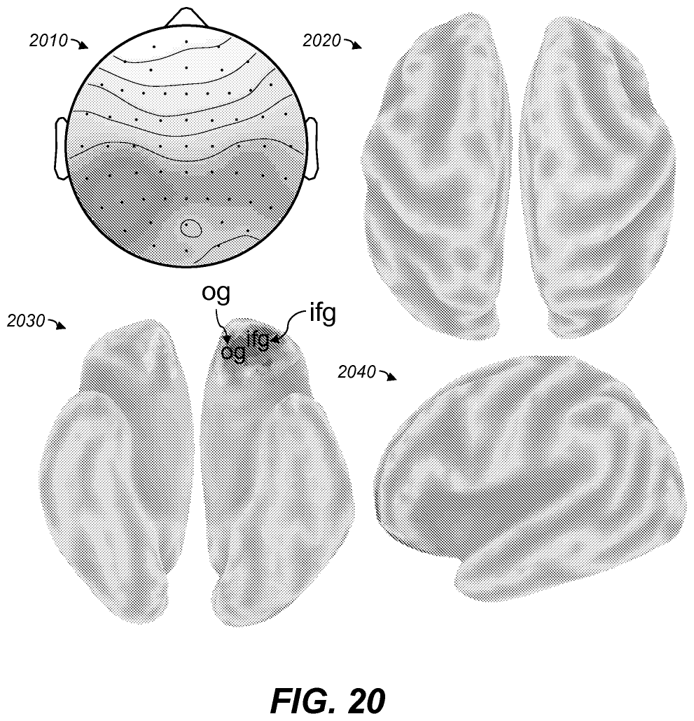

Referring to FIG. 20, the activity captured by component 3 is consistent with sources in the inferior frontal gyrus (ifg) and the orbital gyrus (og).

Referring back to FIG. 18, the localization results from the first component of synchronized activity suggest a possible source in the cingulate cortex, with particularly strong activation occurring in the posterior cingulate of the left hemisphere. The cingulate cortex has been viewed by some as a unitary component of the limbic system subserving emotional processing. Strong activations may also originate in the parahippocampal gyri (involved in the processing of scenes), as well as in the precuneus and superior parietal lobule of the parietal cortex—widespread involvement of the parietal cortex in neural correlation was also reported in fMRI.

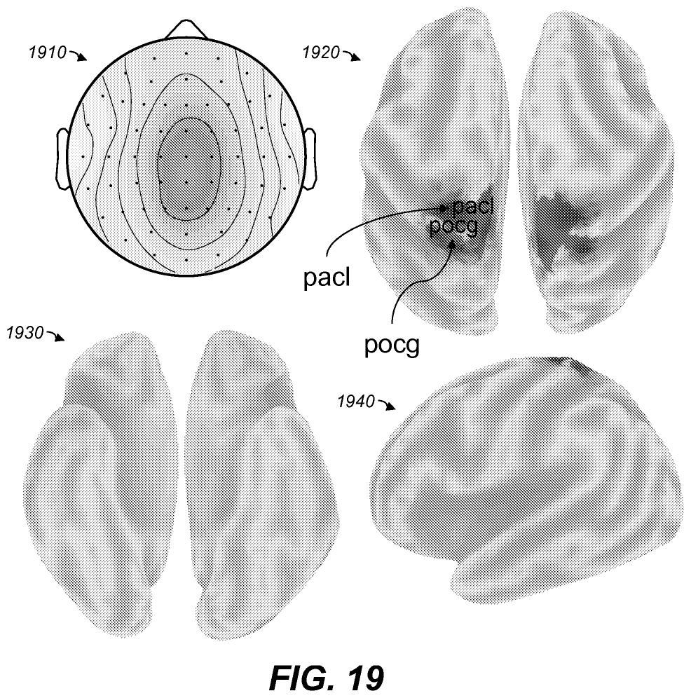

Referring to FIG. 19, performing LORETA on the scalp projection of the synchronized activity in the second component is also consistent with activity originating in the parietal cortex, with the postcentral gyrus and paracentral lobules showing strong activations across both hemispheres.

Referring to FIG. 20, source analysis of activity in the third component reveals possible sources in frontal regions (in descending order of strength of activation): the inferior frontal, orbital, middle frontal, and superior frontal gyri. The orbitofrontal cortex is considered to be a region of multimodal association and is involved in the representation and learning of reinforcers that elicit emotions and conscious feelings.

In order to investigate the relationship between engagement—an everyday phenomenon which can readily be described subjectively—and neural correlation on a temporally fine time scale, a component analysis technique has been developed. This technique yields cleaner estimates of the underlying neural synchrony than that obtained by simply correlating (noisy) EEG data in an electrode-to-electrode fashion. By then manipulating the naturalistic stimulus (for example, by repeating the film or showing it with scrambled scenes), a close correspondence was found between expected engagement and neural correlation. The observed desynchronization of alpha-band activity during times of high neural correlation suggests increased attention during moments of engagement. Indeed, there may be significant overlap between engagement and attention, as both appear to involve a suppression of internally-oriented mental processing with a focus on external stimuli. In addition to increased attention, engagement entails emotional involvement (“emotionally-laden attention”). This is supported by the finding of decreased beta activity. Furthermore, increased theta activity is found in frontal areas; this has been repeatedly implicated in memory encoding. This is also consistent with the finding that the most memorable events are those that are emotionally arousing.

The analysis was repeated but with canonical correlation analysis analysis employed to derive the components. The resulting spatial filters exhibited very noisy topologies with seemingly little anatomical plausibility. This may be due to the higher dimensionality of canonical correlation analysis and insufficient data to fit its parameter space. Both the Correlated Components Analysis (CCA) described herein and the classical canonical correlation analysis explicitly correlate two data sets; instead, one may also apply conventional source separation algorithms such as Independent Components Analysis (ICA) to a concatenated data matrix of the form [X1X2]. Blind source separation techniques such as ICA are also powerful in extracting artifactual components which may then be straightforwardly subtracted from the data. On the other hand, the components yielded by an ICA decomposition are unordered and do not necessarily represent activity that is correlated across viewings. Thus, a manual procedure (and subsequent multiple comparison correction) would be required to search for components which exhibit the desired behavior (i.e., correlation across viewings). To that end, an ICA-type algorithm which incorporates correlation constraints may prove useful in future investigations.

Analyzing naturalistic data presents a challenge in that segments of data severely corrupted by subject movement and rapid impedance changes need to be retained in the processed data set: in multiple-trial analyses of the event-related variety, one may simply discard corrupted trials. In the analyses described herein, to preserve the temporal structure of the data, all samples varying from their channel's mean by more than 4 standard deviations have been replaced with zeros. The obtained components do not show temporal time courses or spatial topologies consistent with motion artifacts. Ultimately, the effects of the manipulations used showing the film a second time or with its scenes scrambled) on the resulting neural correlations suggest that what is being observed is neural in origin.

The analysis of the cortical origins of scalp potentials, particularly in the third component, argues for possible sources in the orbitofrontal cortex associated with emotional involvement. While analysis of scalp potentials cannot conclusively pinpoint the location of a current source in the brain, it can nevertheless suggest which source locations are consistent with the data, and thus helps to generate hypotheses as to the spatial origins of activity. Combined fMRI-EEG experiments can be performed to test the estimates observed here. Moreover, a combined fMRI-EEG study can be performed to ascertain the importance of temporal resolution in identifying moments of high “engagement”—while the frame rate of a film far exceeds the temporal resolution of any fMRI scanner, the information rates of natural audiovisual stimuli are substantially lower than the frame rates employed to display their content. The fine temporal resolution of EEG may allow one to establish the time scale at which engagement is regulated in human subjects—something likely not feasible with fMRI.

Given the rising interest in the workings of the brain under real world conditions, the decoding and tracking of brain states in natural, uncontrolled settings promises to be a vigorous research direction in the coming years. While naturalistic experiments are straightforward to conduct (in contrast to the more controlled variety of event-related designs), the task of analysis becomes substantially more difficult in the sense that discerning the features of the perceptually-rich, unregularized stimuli is a non-trivial undertaking. Results described herein point to the ability of marking ongoing attentional and emotional changes using temporally localized changes in neural synchrony. Moreover, it may be possible to differentiate stimuli eliciting peaks in IaSC with those evoking peaks in ISC. Intuitively, IaSC measures how reliably a scene elicits a response in the viewer in repeated presentations. It is thus not surprising that the respective components were found to correspond to markers of engagement. On the other hand, ISC conveys an agreement of a group of individuals, in that correlation peaks when multiple viewers experience a common stimulus similarly. The within subject correlations were strongly modulated by the “meaning” of the stimuli, in the sense that identical stimuli with a disrupted narrative strongly attenuated IaSC. ISC may similarly depend on narrative. Whether the agreement of the group of individuals expressed by ISC is group specific, i.e. “cultural”, or whether a narrative is universally engaging may be an interesting subject for further study.

From a dynamical systems viewpoint of the brain, sensory processing interrupts internally-oriented “default-mode” activity. Various algorithms herein are used to extract the stimulus-driven response while filtering out the intrinsic activity. In actuality, the neural response to the stimulus varies both within and across subjects due to subjective evaluations of the stimulus, and due to the uniqueness of each individual's brain. Moreover, resting-state activity may exhibit some correlation across viewings. In general, however, projections of the data which maximize correlation across viewings will reflect more of the sensory processing and less of the default-mode activity than that of the raw recordings.

The observed involvement of attention and emotion suggests that cortical processing of external stimuli is modulated by cognitive states. In this view, the brain is a dynamical system in which its extrinsic response to a stimulus is shaped by its global state. For example, the amplitude modulating effect of attention on visual evoked response has been observed as early as the 1960's. Thus, the neural activity of a less attentive viewer will exhibit less of the extrinsic response and more of the intrinsic activity (the effective “noise”), leading to decreased correlation across multiple views. Another possibility is that sensory processing becomes more precisely time-locked to the stimulus during periods of high engagement.

Results described herein demonstrate that the amount of temporally-resolved neural correlation conveys high-level properties of the stimulus.

In view of the foregoing, various aspects provide improved processing of neural data, e.g., for neuromarketing. A technical effect of various aspects is to determine a correlation between measured brain activity of a small group of people and measured behavior of a large group of people.

### CONCLUSION

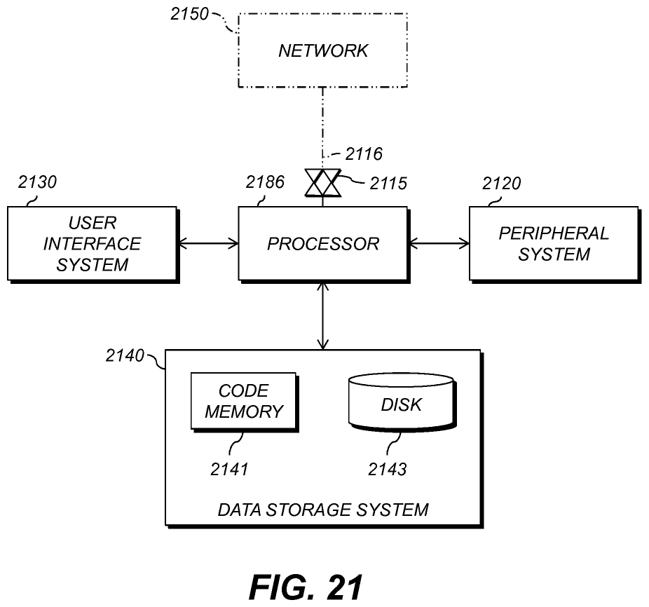

FIG. 21 is a high-level diagram showing the components of an exemplary data-processing system for analyzing data and performing other analyses described herein, and related components. The system includes a processor 2186, a peripheral system 2120, a user interface system 2130, and a data storage system 2140. The peripheral system 2120, the user interface system 2130 and the data storage system 2140 are communicatively connected to the processor 2186. Processor 2186 can be communicatively connected to network 2150 (shown in phantom), e.g., the Internet or an X.215 network, as discussed below. Processor 2186 can include one or more of systems 2120, 2130, 2140, and can each connect to one or more network(s) 2150. Processor 2186, and other processing devices described herein, can each include one or more microprocessors, microcontrollers, field-programmable gate arrays (FPGAs), application-specific integrated circuits (ASICs), programmable logic devices (PLDs), programmable logic arrays (PLAs), programmable array logic devices (PALs), or digital signal processors (DSPs).

Processor 2186 can implement processes of various aspects described herein, e.g., as shown in FIGS. 1 and 2. Processor 2186 can be or include one or more device(s) for automatically operating on data, e.g., a central processing unit (CPU), microcontroller (MCU), desktop computer, laptop computer, mainframe computer, personal digital assistant, digital camera, cellular phone, smartphone, or any other device for processing data, managing data, or handling data, whether implemented with electrical, magnetic, optical, biological components, or otherwise. Processor 2186 can include Harvard-architecture components, modified-Harvard-architecture components, or Von-Neumann-architecture components.

The phrase “communicatively connected” includes any type of connection, wired or wireless, for communicating data between devices or processors. These devices or processors can be located in physical proximity or not. For example, subsystems such as peripheral system 2120, user interface system 2130, and data storage system 2140 are shown separately from the data processing system 2186 but can be stored completely or partially within the data processing system 2186.

The peripheral system 2120 can include one or more devices configured to provide digital content records to the processor 2186. For example, the peripheral system 2120 can include digital still cameras, digital video cameras, cellular phones, or other data processors. The processor 2186, upon receipt of digital content records from a device in the peripheral system 2120, can store such digital content records in the data storage system 2140.

The user interface system 2130 can include a mouse, a keyboard, another computer (connected, e.g., via a network or a null-modem cable), or any device or combination of devices from which data is input to the processor 2186. The user interface system 2130 also can include a display device, a processor-accessible memory, or any device or combination of devices to which data is output by the processor 2186. The user interface system 2130 and the data storage system 2140 can share a processor-accessible memory.

In various aspects, processor 2186 includes or is connected to communication interface 2115 that is coupled via network link 2116 (shown in phantom) to network 2150. For example, communication interface 2115 can include an integrated services digital network (ISDN) terminal adapter or a modem to communicate data via a telephone line; a network interface to communicate data via a local-area network (LAN), e.g., an Ethernet LAN, or wide-area network (WAN); or a radio to communicate data via a wireless link, e.g., WiFi or GSM. Communication interface 2115 sends and receives electrical, electromagnetic or optical signals that carry digital or analog data streams representing various types of information across network link 2116 to network 2150. Network link 2116 can be connected to network 2150 via a switch, gateway, hub, router, or other networking device.

Processor 2186 can send messages and receive data, including program code, through network 2150, network link 2116 and communication interface 2115. For example, a server can store requested code for an application program (e.g., a JAVA applet) on a tangible non-volatile computer-readable storage medium to which it is connected. The server can retrieve the code from the medium and transmit it through network 2150 to communication interface 2115. The received code can be executed by processor 2186 as it is received, or stored in data storage system 2140 for later execution.

Data storage system 2140 can include or be communicatively connected with one or more processor-accessible memories configured to store information. The memories can be, e.g., within a chassis or as parts of a distributed system. The phrase “processor-accessible memory” is intended to include any data storage device to or from which processor 2186 can transfer data (using appropriate components of peripheral system 2120), whether volatile or nonvolatile; removable or fixed; electronic, magnetic, optical, chemical, mechanical, or otherwise. Exemplary processor-accessible memories include but are not limited to: registers, floppy disks, hard disks, tapes, bar codes, Compact Discs, DVDs, read-only memories (ROM), erasable programmable read-only memories (EPROM, EEPROM, or Flash), and random-access memories (RAMs). One of the processor-accessible memories in the data storage system 2140 can be a tangible non-transitory computer-readable storage medium, i.e., a non-transitory device or article of manufacture that participates in storing instructions that can be provided to processor 2186 for execution.

In an example, data storage system 2140 includes code memory 2141, e.g., a RAM, and disk 2143, e.g., a tangible computer-readable rotational storage device such as a hard drive. Computer program instructions are read into code memory 2141 from disk 2143. Processor 2186 then executes one or more sequences of the computer program instructions loaded into code memory 2141, as a result performing process steps described herein, e.g., as shown in FIGS. 1 and 2. In this way, processor 2186 carries out a computer implemented process. For example, steps of methods described herein, blocks of the flowchart illustrations or block diagrams herein, and combinations of those, can be implemented by computer program instructions. Code memory 2141 can also store data, or can store only code.

Various aspects described herein may be embodied as systems or methods. Accordingly, various aspects herein may take the form of an entirely hardware aspect, an entirely software aspect (including firmware, resident software, micro-code, etc.), or an aspect combining software and hardware aspects. These aspects can all generally be referred to herein as a “service,” “circuit,” “circuitry,” “module,” or “system.”

Furthermore, various aspects herein may be embodied as computer program products including computer readable program code stored on a tangible non-transitory computer readable medium. Such a medium can be manufactured as is conventional for such articles, e.g., by pressing a CD-ROM. The program code includes computer program instructions that can be loaded into processor 2186 (and possibly also other processors), to cause functions, acts, or operational steps of various aspects herein to be performed by the processor 2186 (or other processor). Computer program code for carrying out operations for various aspects described herein may be written in any combination of one or more programming language(s), and can be loaded from disk 2143 into code memory 2141 for execution. The program code may execute, e.g., entirely on processor 2186, partly on processor 2186 and partly on a remote computer connected to network 2150, or entirely on the remote computer.

The invention is inclusive of combinations of the aspects described herein. References to “a particular aspect” (or “embodiment” or “version”) and the like refer to features that are present in at least one aspect of the invention. Separate references to “an aspect” or “particular aspects” or the like do not necessarily refer to the same aspect or aspects; however, such aspects are not mutually exclusive, unless so indicated or as are readily apparent to one of skill in the art. The use of singular or plural in referring to “method” or “methods” and the like is not limiting. The word “or” is used in this disclosure in a non-exclusive sense, unless otherwise explicitly noted.

The invention has been described in detail with particular reference to certain preferred aspects thereof, but it will be understood that variations, combinations, and modifications can be effected by a person of ordinary skill in the art within the spirit and scope of the invention.

1. (canceled)

2. A system for predicting a population behavioral response, the system comprising:
data processing hardware communicatively coupled to a plurality of electroencephalographic (EEG) sensors and operable to:
receive neural data from the plurality of EEG sensors capturing brain responses from a first population in response to a media stimulus, wherein the neural data has a first dimensionality;
generate reduced-dimensional neural data that reduces the first dimensionality of the neural data to a mapping dimensionality that is compatible with a second dimensionality of behavioral data used for training a predictive model, wherein the second dimensionality is less than the first dimensionality, wherein the reduced-dimensional neural data is generated using modulated correlated component analysis (CorrCA), and wherein the CorrCA performs operations including:
identifying a first set of time periods and a second set of time periods from the neural data, wherein each time period of the first set includes a window of time where the brain responses have a first level of audience behavioral response, wherein each time period of the second set includes a corresponding window of time where the brain responses have a second level of audience behavior response, and wherein the first level has a degree of response opposite the second level;
for each time period of the first set and the second set, determining a correlation among the brain responses that is proportional to the degree of response for the respective levels of audience behavior response; and
generating the reduced-dimensional neural data having the mapping dimensionality as a time series of the correlations from each time period;
generate a behavioral prediction using the predictive model, wherein the predictive model is configured to receive, as input, the reduced-dimensional neural data and to generate, as output, the behavioral prediction indicating a behavioral response to the media stimulus, and wherein the predictive model has been trained by:
receiving training data representing responses of a second population to a reference media stimulus, wherein the second population includes individuals absent from the first population, and wherein the training data includes behavioral training data and neural training data corresponding to the behavior training data;
reducing the dimensionality of the neural training data to the mapping dimensionality using the operations of the CorrCA;
generating an estimation of the behavioral training data from an input of the neural training data having the mapping dimensionality; and
tuning a set of tunable parameters of the predictive model using the estimation of the behavioral training data and the behavior training data; and
predict, using the behavioral prediction, a population behavioral response to a test media stimulus.

3. The system of claim 2, wherein the data processing hardware automatically divides the reference media stimulus into a plurality of segments.

4. The system of claim 3, wherein the operations of the CorrCA further include:
selecting a respective portion of the neural training data corresponding to each of the segments;
determining a respective neural response reliability for each of the selected portions; and
providing the determined neural response reliabilities as a measure of across-subject agreement.

5. The system of claim 4, wherein each respective determined neural response reliability indicates a consistency between respective neurological responses of at least two subjects in the second population to the corresponding segment.

6. The system of claim 2, wherein the population response indicates a predicted action taken by a subject in response to exposure to the test media stimulus.

7. The system of claim 2, wherein the second population has more members than the second population.

8. The system of claim 2, wherein the behavioral training data includes communication via social media.

9. The system of claim 2, wherein at least one of the reference media stimulus or the test media stimulus include a set of advertisements.

10. The system of claim 2, wherein at least one of the reference media stimulus or the test media stimulus is selected from a group consisting of a news broadcast, a TV or radio program, a movie, a piece of music, and an instructional video.

11. The system of claim 2, wherein the time series of the reduced-dimensional neural data has a lower temporal resolution than the neural data from the plurality of EEG sensors.

12. A method comprising:
receiving, at data processing hardware, neural data from a plurality of electroencephalographic (EEG) sensors capturing brain responses from a first population in response to a media stimulus, wherein the neural data has a first dimensionality;
generating, by the data processing hardware, reduced-dimensional neural data that reduces the first dimensionality of the neural data to a mapping dimensionality that is compatible with a second dimensionality of behavioral data used for training a predictive model, wherein the second dimensionality is less than the first dimensionality, wherein the reduced-dimensional neural data is generated using modulated correlated component analysis (CorrCA), and wherein the CorrCA performs operations including:
identifying a first set of time periods and a second set of time periods from the neural data, wherein each time period of the first set includes a window of time where the brain responses have a first level of audience behavioral response, wherein each time period of the second set includes a corresponding window of time where the brain responses have a second level of audience behavior response, and wherein the first level has a degree of response opposite the second level;
for each time period of the first set and the second set, determining a correlation among the brain responses that is proportional to the degree of response for the respective levels of audience behavior response; and
generating the reduced-dimensional neural data having the mapping dimensionality as a time series of the correlations from each time period;
generating, by the data processing hardware, a behavioral prediction using the predictive model, wherein the predictive model is configured to receive, as input, the reduced-dimensional neural data and to generate, as output, the behavioral prediction indicating a behavioral response to the media stimulus, and wherein the predictive model has been trained by:
receiving training data representing responses of a second population to a reference media stimulus, wherein the second population includes individuals absent from the first population, and wherein the training data includes behavioral training data and neural training data corresponding to the behavior training data;
reducing the dimensionality of the neural training data to the mapping dimensionality using the operations of the CorrCA;
generating an estimation of the behavioral training data from an input of the neural training data having the mapping dimensionality; and
tuning a set of tunable parameters of the predictive model using the estimation of the behavioral training data and the behavior training data; and
predicting, by the data processing hardware using the behavioral prediction, a population behavioral response to a test media stimulus.

13. The method of claim 12, further including automatically dividing, by data processing device, the reference media stimulus into a plurality of segments.

14. The method of claim 13, wherein the operations of the CorrCA further include:
selecting a respective portion of the neural training data corresponding to each of the segments;
determining a respective neural response reliability for each of the selected portions; and
providing the determined neural response reliabilities as a measure of across-subject agreement.

15. The method of claim 14, wherein each respective determined neural response reliability indicates a consistency between respective neurological responses of at least two subjects in the second population to the corresponding segment.

16. The method of claim 13, wherein the population response indicates a predicted action taken by a subject in response to exposure to the test media stimulus.

17. The method of claim 12, wherein the second population has more members than the second population.

18. The method of claim 12, wherein the behavioral training data includes communication via social media.

19. The method of claim 12, wherein at least one of the reference media stimulus or the test media stimulus include a set of advertisements.

20. The method of claim 12, wherein at least one of the reference media stimulus or the test media stimulus is selected from a group consisting of a news broadcast, a TV or radio program, a movie, a piece of music, and an instructional video.

21. The method of claim 12, wherein the time series of the reduced-dimensional neural data has a lower temporal resolution than the neural data from the plurality of EEG sensors.
